SEQUENCE LISTING (22 sequences)

SEQ ID NO: 1 - protein, 15 aa, Artificial Sequence
  Peptide found in the Crf1 protein of Aspergillus fumigatus
HTYTIDWTKDAVTWS

SEQ ID NO: 2 - protein, 15 aa, Artificial Sequence
  Peptide found in the Crh1 protein of Candida albicans
HTYVIDWTKDAVTWS

SEQ ID NO: 3 - protein, 11 aa, Artificial Sequence
  peptide epitope of A. fumigatus Crf1
HTYVIDWTKDA

SEQ ID NO: 4 - protein, 15 aa, Artificial Sequence
  peptide of A. fumigatus Crf1
FHTYTIDWTKDAVTW

SEQ ID NO: 5 - protein, 15 aa, Artificial Sequence
  peptide of A. fumigatus Crf1
PVATPQETFHTYTID

SEQ ID NO: 6 - protein, 15 aa, Artificial Sequence
  peptide of A. fumigatus Crf1
PQETFHTYTIDWTKD

SEQ ID NO: 7 - protein, 15 aa, Artificial Sequence
  peptide of A. fumigatus Crf1
TIDWTKDAVTWSIDG

SEQ ID NO: 8 - protein, 15 aa, Artificial Sequence
  peptide of A. fumigatus Crf1
TKDAVTWSIDGAVVR

SEQ ID NO: 9 - protein, 11 aa, Artificial Sequence
  peptide of A. fumigatus Crf1
HTYTIDWTKDA

SEQ ID NO: 10 - protein, 11 aa, Artificial Sequence
  11-mer peptide of p40/41
QETFHTYTIDW

SEQ ID NO: 11 - protein, 11 aa, Artificial Sequence
  11-mer peptide of p40/41
TFHTYTIDWTK

SEQ ID NO: 12 - protein, 15 aa, Artificial Sequence
  11-mer peptide of p40/41
YTMYVKSVRIENANP

SEQ ID NO: 13 - protein, 15 aa, Artificial Sequence
  peptide epitope of A. fumigatus Crf1
GSSNTGSGSGSGSGS

SEQ ID NO: 14 - protein, 15 aa, Artificial Sequence
  peptide epitope of A. fumigatus Crf1
MYFKYTAAALAAVLP

SEQ ID NO: 15 - protein, 15 aa, Artificial Sequence
  peptide epitope of A. fumigatus Crf1
TRFPQTPMRLRLGSW

SEQ ID NO: 16 - protein, 15 aa, Artificial Sequence
  peptide epitope of A. fumigatus Crf1
TGSSTSAGASATPEL

SEQ ID NO: 17 - protein, 15 aa, Artificial Sequence
  peptide epitope of A. fumigatus Crf1
PPNKGLAASTYTADF

SEQ ID NO: 18 - protein, 15 aa, Artificial Sequence
  peptide epitope of A. fumigatus Crf1
AKGGTRFPQTPMRLR

SEQ ID NO: 19 - protein, 15 aa, Artificial Sequence
  peptide epitope of A. fumigatus Crf1
SAGPYTMYVKSVRIE

SEQ ID NO: 20 - protein, 15 aa, Artificial Sequence
  peptide epitope of A. fumigatus Crf1
SSSSVTSSTTSTASS

SEQ ID NO: 21 - protein, 395 aa, Aspergillus fumigatus
MYFKYTAAALAAVLPLCSAQTWSKCNPLEKTCPPNKGLAASTYTADFTSASALDQWEVTAGKVPVGPQGAEFTVAKQGDAPTIDTDFYFFFGKAEVVMKAAPGTGVVSSIVLESDDLDEVDWEVLGGDTTQVQTNYFGKGDTTTYDRGTYVPVATPQETFHTYTIDWTKDAVTWSIDGAVVRTLTYNDAKGGTRFPQTPMRLRLGSWAGGDPSNPKGTIEWAGGLTDYSAGPYTMYVKSVRIENANPAESYTYSDNSGSWQSIKFDGSVDISSSSSVTSSTTSTASSASSTSSKTPSTSTLATSTKATPTPSGTSSGSNSSSSAEPTTTGGTGSSNTGSGSGSGSGSGSSSSTGSSTSAGASATPELSQGAAGSIKGSVTACALVFGAVAAVLAF

SEQ ID NO: 22 - protein, 269 aa, Candida albicans
KTLTISTLFAANANADTKSSDSVPAGSFLEKDNGLGPHFSLKKQTIDSSNLSLMKRFNSFKSNIMRVLGEKIFYQIIMFPYWSINAGHDINLKDYVVSIIPKDNQSAIYAIQPDIQAFGILVADYSSGKQSQEAGKVNGRYDQAQDDIKKLESGQSVDNSPASDSSTSSSSPSTSSSSSSSSSSSSSEKNVSASVIVFIGKDKTTVSGVSVPTSASVSAATTANSAPAAAASTVFISTGDAAPSSEKSVTTENNAVAKTTSLFLIGFFV

(19) United States
(12) Patent Application Publication
(10) Pub. No.: US 2013/0315920 A1
(43) Pub. Date: Nov. 28, 2013

(54) Cross Protective Epitopes of Aspergillus Fumigatus and Candida Albicans

(71) Applicants: Max S. Topp, Wuerzburg (DE); Claudia Stuehler, Basel (CH); Nina Khanna, Basel (CH)

(72) Inventors: Max S. Topp, Wuerzburg (DE); Claudia Stuehler, Basel (CH); Nina Khanna, Basel (CH)

(73) Assignee: Cell Medica Limited, London (GB)

(21) Appl. No.: 13/988,036

(22) PCT Filed: Nov. 16, 2011
(86) PCT No.: PCT/GB2011/052239

§ 371 Date: Aug. 6, 2013

(30) Foreign Application Priority Data
Nov. 17, 2010 (GB) 1019437.1

(51) Int. Cl. C07K 7/08; G01N 33/68; C07K 14/74

(52) U.S. Cl. CPC C07K 7/08; C07K 14/70539; G01N 33/6893
USPC 424/139.1; 530/405; 530/350; 536/23.5; 435/320.1; 435/325; 424/93.71; 424/93.21; 530/387.9; 435/7.24; 424/185.1; 514/3.4; 435/69.1; 514/21.5; 514/44 R; 424/184.1; 435/455; 514/3.3

(57) ABSTRACT

A complex comprising a Class II HLA-DRB1*03 or Class II HLA-DRB1*13 molecule bound to a peptide, wherein the peptide comprises the amino acid sequence HTYTIDWTKDAVTWS or a portion thereof, or a variant of the given amino acid sequence or portion wherein the side chains of one or two or three or four or five or six or seven of the amino acid residues are altered, wherein the peptide comprising the portion, or variant, is capable of binding HLA-DRB1*03 and/or HLA-DRB1*13. The complex may be used to select Aspergillus and Candida antigen-specific T cells. A vaccine against Candida albicans infection comprising (a) a peptide comprising the amino acid sequence HTYTIDWTKDAVTWS or a portion thereof, or a variant of the given amino acid sequence or portion wherein the side chains of one or two or three or four or five or six or seven of the amino acid residues are altered, wherein the peptide comprising the portion, or variant, is capable of binding HLA-DRB1*03 and/or HLA-DRB1*04 and/or HLA-DRB1*13, or (b) a polypeptide which is a fusion of any of the peptides of (a) and another peptide, or (c) a polynucleotide encoding a peptide of (a) or a polypeptide of (b) or (d) an expression vector capable of expressing a peptide of (a) or a polypeptide of (b). A peptide of less than 10,000 molecular weight comprising the amino acid sequence HTYTIDWTKDAVTWS or a portion thereof or a variant of the given amino acid sequence or portion wherein the side chains of one or two or three or four or five or six or seven of the amino acid residues are altered, wherein the peptide comprising the portion, or variant, is capable of binding HLA-DRB1*03 and/or HLA-DRB1*13.

The present invention relates to immunotherapeutic methods, and molecules and cells for use in immunotherapeutic methods. In particular, the present invention relates to the immunotherapy of Aspergillus and Candida albicans infection, including adoptive immunotherapy.

The listing or discussion of an apparently prior-published document in this specification should not necessarily be taken as an acknowledgement that the document is part of the state of the art or is common general knowledge.

The saprophytic mold Aspergillus fumigatus and Candida albicans, a commensal of the mucocutaneous membranes are the most common fungal pathogens among immunocompromised patients. Both fungi cause severe invasive infections and are responsible for substantial morbidity and mortality despite effective antifungal treatment¹. Immunotherapeutic approaches are promising to enhance the pathogen-specific immune system and thereby limit infectious complications. Previously, adoptively transferred A. fumigatus-specific T_{H}1 clones generated using heat-inactivated fungus as antigen were capable of ameliorating invasive aspergillosis (IA) without causing adverse effects in patients after hematopoietic stem cell transplantation (HSCT)². The beneficial effect of adoptive T-cell therapy could be increased if the transferred T-cells were reactive not only to A. fumigatus but to other fungal species as well. In spite of genetic and morphologic disparities and distinct clinical diseases, the host immunity to different fungi is quite similar. In IA as well as in invasive candidiasis T_{H}1-biased immune responses correlate with protective immunity and resistance, whereas T_{H}2-biased responses generally lead to an exacerbation of disease³⁻⁶. T_{H}1 cell activation is instrumental to clear the infection by improving the effector function of innate immune cells through the release of pro-inflammatory cytokines⁴.

Previous data in mice have suggested the existence of cross-protective immunity between different fungal species. For instance, vaccination with Saccharomyces yeast was able to protect mice against subsequent lethal infection with a number of different fungi^{7,8}. The mechanism of this cross-protection is not yet fully elucidated and could be based either on unspecific immunostimulatory effects indiscriminately enhancing the effector function of innate immune cells or on adaptive immunity triggering antigen-specific antibody- or T-cell-mediated responses. Some different fungal species share conserved gene products to a certain degree and sometimes T-cell receptor (TCR) binding degeneracy allows recognition of several similar peptides by a single TCR⁹. We sought to determine whether A. fumigatus antigens can induce T_{H}1 cells with cross-reactivity to other fungal species in humans and mice. Surprisingly, we have identified that a peptide epitope (and related peptides) of the Aspergillus fumigatus Crf1 protein not only gives rise to protection against Aspergillus, but also gives rise to protection against the unrelated yeast Candida albicans. In addition, the work described herein also surprisingly reveals that the peptide epitope (and related peptides) is not restricted to presentation by the Class II molecule HLA-DRB1*04 (see WO 2009/112831); rather, it may also be presented by the Class II molecules HLA-DRB1*03 and/or HLA-DRB1*13. These finding provide for further methods of combating Aspergillus and Candida albicans infection and allergy. The Crf1 protein has also been called the Asp f16 antigen.

Human diseases associated with Aspergillus include Allergic Bronchopulmonary Aspergillosis (ABPA), Invasive Aspergillosis (IA), Aspergilloma, and Chronic Aspergillus sinusitis. ABPA is caused by the presence of Aspergillus in the bronchopulmonary region leading to inflammation and allergic responses. Bronchiectasis and long-term lung degeneration develops with symptoms similar to asthma. Invasive Aspergillosis is associated with immune deficiencies. The infection usually begins in the lungs and may transfer to other organs. The mortality rate associated with IA is high. Aspergilloma results when other conditions create cavities in the lung which allow the Aspergillus spores to germinate and to colonize the area, forming a fungal ball. The disease is often chronic and incurable. Chronic Aspergillus sinusitis develops in the sinus in a similar manner to aspergilloma in the lungs. There is increasing belief that many chronic sinus problems are a result of allergic reactions to fungus.

Human diseases associated with Candida albicans include cutaneous candidiasis syndromes, chronic mucocutaneous candidiasis, gastrointestinal tract candidiasis, genitourinary tract candidiasis, respiratory tract candidiasis, hepatosplenic candidiasis and systemic candidiasis.

T-cell mediated heterologous immunity to different pathogens is promising for the development of immunotherapeutic strategies. Aspergillus fumigatus and Candida albicans, the two most common fungal pathogens causing severe infections in immunocompromised patients, are controlled by CD4⁺ T_{H}1 cells in humans and mice, making induction of fungus-specific CD4⁺ T_{H}1 immunity an appealing strategy for antifungal therapy. We identified an immunogenic epitope of the A. fumigatus cell wall glucanase Crf1 that can be presented by three common MHC class II alleles and induces memory CD4⁺ T_{H}1 cells with a diverse T-cell receptor repertoire that are cross-reactive to C. albicans. In BALB/c mice, the Crf1 protein also elicits cross-protection against lethal infection with C. albicans that is mediated by the same epitope as in humans. This data illustrates the existence of T-cell based cross-protection for the two distantly related clinically relevant fungal pathogens.

Aspergillus and Candida albicans antigen-specific CD4⁺ T cells are useful in adoptive T cell therapy which may be of benefit in patients with evidence of Aspergillus- or Candida albicans-related diseases, or for prophylactic or preemptive therapy, for example in asymptomatic HSCT recipients. The discovery of the wider HLA restriction peptide epitope allows the identification of further specific patients that can benefit from the use of this peptide in immunotherapy protocols for combating infection and allergy. The discovery of the protective effect in relation to Candida albicans allows for new approaches in combating Candida albicans infection and allergy.

Surprisingly, the peptide epitope (and related peptides) may be presented by Class II HLA-DRB1*03 or HLA-DRB1*13 as well as HLA-DRB1*04. These three haplotypes are found in >50% of the human population.

A first aspect of the invention provides a complex comprising a Class II HLA-DRB1*03 or Class II HLA-DRB1*13 molecule bound to a peptide, wherein the peptide comprises the amino acid sequence HTYTIDWTKDAVTWS or a portion thereof, or a variant thereof wherein the side chains of one or two or three or four or five or six or seven of the amino acid residues are altered, wherein the peptide comprising the portion, or variant, is capable of binding HLA-DRB1*03 and/or HLA-DRB1*13.

The peptide HTYTIDWTKDAVTWS is described as p41 in Example 1. Preferably, the complex contains a peptide consisting of the amino acid sequence HTYVIDWTKDAVTWS.

As is discussed in more detail below, the complexes of this aspect of the invention are useful for identifying, selecting and isolating T cells specific for the bound peptide. As is disclosed in Example 1, the peptide, when presented by a suitable Class II molecule, can identify antigen specific T cells which we have termed “Aspergillus and Candida antigen-specific T cells”. The T cells may also be considered to be Crf1 peptide (or p41)-specific but as explained in Example 1 the p41 peptide is found in the Crf1 protein of Aspergillus fumigatus whereas the closely related peptide HTYVIDWTKDAVTWS is found in the Crh1 protein of Candida albicans and the T cells are reactive to both peptides.

The specific peptide with the amino acid sequence HTYTIDWTKDAVTWS has been shown to bind to HLA-DRB1*03, HLA-DRB1*13 and HLA-DRB1*04.

HLA-DRB1*03 includes the sub-types DRB1*03010101; DRB1*030102; DRB1*030201; DRB1*030202; DRB1*0303; DRB1*0304; DRB1*030501; DRB1*030502; DRB1*0306; DRB1*0307; DRB1*0308; DRB1*0309; DRB1*0310; DRB1*0311; DRB1*0312; DRB1*0313; DRB1*0314; DRB1*0315; DRB1*0316; DRB1*0317; DRB1*0318; DRB1*0319; DRB1*0320; DRB1*0321; DRB1*0322; DRB1*0323. For the avoidance of doubt we include all of these in the term DRB1*03.

HLA-DRB1*13 includes the sub-types DRB1*130101; DRB1*130102; DRB1*130103; DRB1*130201; DRB1*130202; DRB1*130301; DRB1*130302; DRB1*1304; DRB1*130501; DRB1*130502; DRB1*1306; DRB1*130701; DRB1*130702; DRB11308; DRB1*1309; DRB1*1310; DRB1*131101; DRB1*1312; DRB1*1313; DRB1*131401; DRB1*131402; DRB1*1315; DRB1*1316; DRB1*1317; DRB1*1318; DRB1*1319; DRB1*1320; DRB1*1321; DRB1*1322; DRB1*1323; DRB1*1324; DRB1*1325; DRB1*1326; DRB1*1327; DRB1*1328; DRB1*1329; DRB1*1330; DRB1*1331; DRB1*1332; DRB1*1333; DRB1*1334; DRB1*1335; DRB1*1336; DRB1*1337; DRB1*1338; DRB1*1339; DRB1*1340; DRB1*1341; DRB1*1342; DRB1*1343; DRB1*1344; DRB1*1345; DRB1*1346; DRB1*1347; DRB1*1348; DRB1*1349; DRB1*135001; DRB1*1351; DRB1*1352; DRB1*1353; DRB1*1354; DRB1*1355; DRB1*1356; DRB1*1357; DRB1*1358; DRB1*1359; DRB1*1360; DRB1*1361; DRB1*1362; DRB1*1363; DRB1*1364; DRB1*1365; DRB1*1366; DRB1*1367; DRB1*1368; DRB1*1369; DRB1*1370; DRB1*1371. For the avoidance of doubt we include all of these in the term DRB1*13.

HLA-DRB1*04 includes the sub-types DRB1*040101; DRB1*040102; DRB1*0402; DRB1*040301; DRB1*040302; DRB1*0404; DRB1*040501; DRB1*040502; DRB1*040503; DRB1*040504; DRB1*040601; DRB1*040602; DRB1*040701; DRB1*040702; DRB1*040703; DRB1*0408; DRB1*0409; DRB1*0410; DRB1*0411; DRB1*0412; DRB1*0413; DRB1*0414; DRB1*0415; DRB1*0416; DRB1*0417; DRB1*0418; DRB1*0419; DRB1*0420; DRB1*0421; DRB1*0422; DRB1*0423; DRB1*0424; DRB1*0425; DRB1*0426; DRB1*0427; DRB1*0428; DRB1*0429; DRB1*0430; DRB1*0431; DRB1*0432; DRB1*0433; DRB1*0434; DRB1*0435; DRB1*0436; DRB1*0437 DRB1*0438; DRB1*0439; DRB1*0440; DRB1*0441; DRB1*0442; DRB1*0443 DRB1*0444; DRB1*0445; DRB1*0446; DRB1*0447; DRB1*0448; DRB1*0449; DRB1*0450; DRB1*0451; DRB1*0452; DRB1*0453; DRB1*0454; DRB1*0455; DRB1*0456. For the avoidance of doubt we include all of these in the term DRB1*04. The relevance of HLA-DRB1*04 is discussed in more detail below with respect to other aspects of the invention.

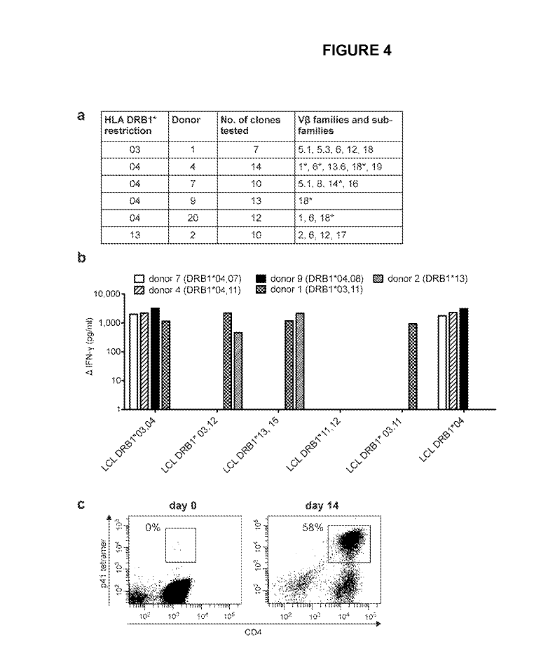

Whether or not a peptide binds to HLA-DRB1*03 or HLA-DRB1*13 or HLA-DRB1*04 may be determined using any method known in the art. A suitable method is shown in Example 1, which uses stimulation with peptide-pulsed partially matched LCLs (see also FIG. 4). One method is to introduce the peptide into PBMCs taken from an HLA-DRB1*03 (or HLA-DRB1*13 or HLA-DRB1*04) human and determine whether an activating or proliferative response is obtained which is indicative of binding.

The recognition of harmful pathogens or disease causing mutations within self tissue occurs through two mechanisms within the human immune system. Antibody molecules expressed by B cells bind with biological molecules, typically expressed on the surface of invading microorganisms or deviant self cells, in highly specific manner and will label these molecules in a manner which will trigger an appropriate immune response. In addition to the antibody response, pathogens and disease causing mutations can be detected due to unique proteins expressed by the pathogens or mutations. These proteins are broken down into small peptide fragments by natural and continuous protein degradation systems in the human cell. The peptide fragments will bind with special molecules, referred to as HLA Class I and HLA Class II molecules, expressed on the surface of all cells. In the human, these molecules are referred to as HLA molecules and are numbered according the large number of alleles which exist across the human population.

In general in the human, the peptide fragments derived from pathogens bind with specific HLA molecules and are transported to the surface of the cell. The peptides are captured in a particular configuration in a peptide-HLA complex which allows the peptides to be detected by T cell receptors (“TCRs”) expressed on the surface of T cells. Through natural selection and development processes which are linked to the immune system's ability to detect danger signals in connection with the presence of a foreign organism, the human body produces T cells with TCRs which can recognize and distinguish between peptide fragments derived from a harmful pathogen and peptides which are derived from harmless microorganisms or healthy self tissue.

HLA Class 1 molecules present peptides derived mainly from proteins found within the cell to CD8+ T cells, also referred to as cytotoxic T cells or CTL. The peptides which bind with HLA Class I molecules are usually 8-10 amino acids in length. HLA Class II molecules present peptides derived from proteins or organisms which have been endocytosed from the extracellular milieu. HLA Class II molecules present peptides to CD4+ T cells, also referred to as T helper cells although CD4+ T cells may also have direct cytotoxic functions. The peptides which bind to HLA Class II molecules are relatively unconstrained in terms of length, although Class II peptides generally fall within a range of 9-25 amino acids, for example 9 or 10 or 11 or 12 or 13 or 14 or 15 or 16 or 17 or 18 or 19 or 20 or 21 or 22 or 23 or 24 or 25 amino acids.

By “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Mézière et al (1997) J. Immunol. 159, 3230-3237, incorporated herein by reference. This approach involves making pseudopeptides containing changes involving the backbone, and not the orientation of side chains. Mézière et al (1997) show that, at least for HLA class II and T helper cell responses, these pseudopeptides are useful. Retro-inverse peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity of a peptide bond.

It will be appreciated that the peptide may conveniently be blocked at its N- or C-terminus so as to help reduce susceptibility to exoproteolytic digestion. Similarly, it will be appreciated that the peptide of the invention may be in salt form or may contain additional esters of —OH or —COOH groups or amides of —NH₂ groups.

By a “portion” of the given amino acid sequence we mean at least 11 or 12 or 13 or 14 consecutive amino acids of the given sequence such that the peptide containing the portion and preferably the portion itself, is still able to bind to the HLA molecule in substantially the same way as a peptide consisting of the given amino acid sequence or the peptide HTYTIDWTKDAVTWS. Preferably, the peptide comprising the portion is able to bind to the HLA molecule in substantially the same way as a peptide consisting of the given amino acid sequence. A particularly preferred portion is the amino acid sequence HTYTIDWTKDA which has found to be the minimal epitope. It is particularly preferred if peptides of the invention contain this minimal epitope. A particularly preferred portion is the amino acid sequence HTYVIDWTKDA. The peptides of the invention may suitably include this amino acid sequence.

By a “variant” of the given amino acid sequence we mean that the side chains of, for example, one or two or three or four or five or six or seven, preferably one or two or three or four, more preferably one or two, still more preferably one of the amino acid residues of the peptide sequence HTYTIDWTKDAVTWS or portion thereof are altered (for example by replacing them with the side chain of another naturally occurring amino acid residue or some other side chain) such that the peptide is still able to bind to the HLA molecule in substantially the same way as a peptide consisting of the given amino acid sequence. For example, a peptide may be modified so that it at least maintains, if not improves, the ability to interact with and bind the relevant HLA molecule, and so that it at least maintains, if not improves, the ability to generate activated CD4⁺ T cells which can recognise Aspergillus fumigatus. Typically, the amino acid alternatives are conservative in nature, such as from within the groups Gly, Ala; Ile, Leu, Val; Ser, Thr; Tyr, Phe, Trp; Glu, Asp; Gln, Asn, H is, Met, Cys, Ser. A particularly preferred variant is HTYVIDWTKDAVTWS which is found in the Candida albicans Crh1 protein.

Peptides of at least 11 amino acids are preferred for complex formation. Thus, the invention also includes complexes which have bound peptides of 11 or 12 or 13 or 14 or 15 or 16 or 17 or 18 or 19 or 20 amino acids that contain the amino acid sequence HTYTIDWTKDAVTWS. As noted above, the peptides in the complex of this aspect of the invention are capable of binding HLA-DRB1*03 and/or HLA-DRB1*13, and they may also bind HLA-DRB1*04.

Those amino acid residues that are not essential to interact with the T cell receptor can be modified by replacement with another amino acid whose incorporation does not substantially affect T cell reactivity and does not eliminate binding to the relevant HLA allele.

Thus, the peptides of the complex of the invention and in particular ones which are close variants of the peptide HTYTIDWTKDAVTWS, are ones which, typically, selectively and reversibly bind HLA-DRB1*03 and/or HLA-DRB1*13, preferably with high affinity.

It is further preferred that the complexes of the invention are ones which can be used to generate peptide-specific CD4⁺ T cells which mediate specific killing of Aspergillus fumigatus and in particular germinating A. fumigatus conidia and hyphae either through direct cytotoxic effector functions or through enhancement of innate immune functions. Similarly, it is preferred that the complexes of the invention are ones which can be used to generate peptide-specific CD4⁺ T cells which mediate specific killing of Candida albicans.

The complexes of the invention are particularly useful for selecting and defining appropriate T cells, and trace them once they are put into the patient, as discussed below.

It is particularly preferred that in all the immunotherapeutic methods of the invention that the patient to be treated is one who carries Class II HLA-DRB1*03 and/or Class II HLA-DRB1*13 (ie has a Class II HLA-DRB1*03-positive genotype and/or a Class II HLA-DRB1*13-positive genotype), and has antigen presenting cells which express HLA-DRB1*03 and/or HLA-DRB1*13. The HLA genotype or status of a patient can be readily determined prior to treatment by methods known in the art.

The complex of the invention, when present on the surface of a suitable antigen-presenting cell, is capable of eliciting a T cell mediated immune response which mediates or helps to mediate the immune systems attack on Aspergillus and/or Candida albicans. In particular, the production of cytokines by a CD4⁺ T cell may mediate the attack on Aspergillus and/or Candida albicans.

A particular preferred complex of this aspect of the invention is one which contains the peptide HTYTIDWTKDAVTWS. Another preferred complex of the invention of the invention is one in which the peptide is a portion of the peptide having the amino acid sequence HTYTIDWTKDAVTWS, the portion having 11 or 12 or 13 or 14 contiguous amino acids of said sequence, provided that the said portion is capable of binding HLA-DRB1*03 and/or HLA-DRB1*03 or HLA-DRB1*13.

Another preferred complex of this aspect of the invention is one which contains a peptide which is a portion of the peptide having the amino acid sequence HTYTIDWTKDAVTWS, the portion having 11 or 12 or 13 or 14 contiguous amino acids of said sequence except that one or two or three or four or five or six or seven of the amino acids are replaced with another amino acid, providing that the peptide is capable of binding HLA-DRB1*03 and/or HLA-DRB1*13. Typically, one or two or three or four, such as one or two, particularly one of the amino acids are replaced with another amino acid.

If a peptide which is greater than around 15 amino acid residues is used directly to bind to a Class II HLA molecule in the complex of the invention, it is preferred that the residues that flank the core HLA binding region are ones that do not substantially affect the ability of the peptide to bind to the HLA molecule or to present the peptide to an Aspergillus and Candida antigen-specific T cell.

Peptides (at least those containing peptide linkages between amino acid residues) may be synthesised using any method well known in the art, for example by the Fmoc-polyamide mode of solid-phase peptide synthesis as disclosed by Lu et al (1981) J. Org. Chem. 46, 3433 and references therein. Reagents for peptide synthesis are generally available from Calbiochem-Novabiochem (UK) Ltd, Nottingham NG7 2QJ, UK as well as many other commercial providers of chemical and biological reagents. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

The complex of the invention may be comprised in an antigen presenting cell. For example, the complex may be made by loading suitable peptides (such as the HTYTIDWTKDAVTWS peptide) into HLA molecules within an antigen presenting cell, such as a dendritic cell, which is known to contain a Class II HLA-DRB1*03 or HLA-DRB1*13 molecule.

Preferably, the complex is a soluble complex.

Preferably, the Class II HLA-DRB1*03 or HLA-DRB1*13 molecule within the complex is a synthetic molecule.

As is discussed in more detail below, these peptides may be used to create HLA multimers whereby the peptide is able to bind to and therefore stabilises the specific HLA-DRB1*03 and/or HLA-DRB1*03 molecules of the complex of the invention. This enables the HLA molecule to be multimerised and conjugated to a fluorescent molecule or a magnetic bead. This multimer can then recognise and bind directly to an Aspergillus or Candida albicans antigen-specific T cell via the TCR. By using this technique cells can be directly selected from fresh or cultured cells or enumerated in both cell therapy products and in blood samples from patients having received a cell therapy product. It is impossible to use this HLA multimer technology only with knowledge of the peptide, as the T cell will only bind to the peptide which is displayed in the context of the correct HLA molecule.

The complex may be used for identifying Aspergillus and Candida antigen specific T cells. The complex is able to bind selectively to these cells and can be used for selection, isolation and purification of the cells. The complex may also be used to identify an Aspergillus and Candida antigen-specific T cell in a sample.

The invention also includes a method for selecting Aspergillus and Candida antigen-specific T cells, the method comprising contacting a population of T cells with a complex according to the first aspect of the invention. In one embodiment, the complex is present on the surface of an antigen-presenting cell, for example which is present in PBMC. In another embodiment, the Class II HLA molecule in the complex is a synthetic, soluble Class II HLA molecule. The method may also include the further step of isolating the T cell.

Conveniently, the peptide antigen is loaded into Class II HLA-DRB1*03 or HLA-DRB1*13 molecules expressed on the surface of a suitable antigen-presenting cell by contacting a sufficient amount of the peptide, for example the peptide HTYTIDWTKDAVTWS, with an antigen-presenting cell in the absence of other peptides which may complete in binding the target HLA allele. Conveniently, the antigen presenting cell may be transfected with a polynucleotide or expression vector which encodes a peptide (as defined with respect to the peptide present in the complex of the invention), and the cell machinery loads suitable peptides into the class II HLA molecules for presentation.

Preferably, the Aspergillus and Candida antigen-specific T cells are CD4⁺, such as CD4⁺ T_{H}1 cells, or are T regulatory cells (which are particularly relevant for combating allergy) T_{H}1 helper cells may be converted to T regulatory cells by exposure to a CD28 antibody (superagonist) CD8⁺ cells. Typically, then, the cells are CD3⁺ CD4⁺ and/or CD25⁺ and/or Foxp3⁺ and/or GITR⁺ and/or CD127⁺.

In one embodiment the antigen is linked to the HLA Class II molecule expressed on the surface of the antigen presenting cell with a suitable flexible linker such that the peptide can occupy the HLA Class II binding groove, or alternatively the antigen is linked to the Invariant Chain which stabilises the HLA Class II molecule and is involved in directing the molecule through the endosomal pathway common to Class II presentation of peptides. Thus, the antigen presenting cells may be cells which contain a polynucleotide (eg expression vector) encoding the fusion polypeptide discussed above.

The antigen presenting cell may be genetically engineered to ensure the expression of the HLA DRB1*03 and/or HLA-DRB1*13 molecule on its own or in combination with the peptides which form part of the complex or polypeptides which may be processed by the antigen presenting cells into peptides which form part of the complex. The antigen-presenting cells may also express co-stimulatory molecules which are useful in enhancing the immunogenicity of the peptide and the resulting ability to produce Aspergillus and Candida antigen-specific T cells. The antigen-presenting cells may be exposed to various cytokines such as IL-2, IFN-γ, and TNF-α, which are known to activate T cells and to direct a T_{H}1 response.

Suitable methods for selecting Aspergillus and Candida antigen-specific T cells include the use of ELISPOT analysis to confirm the responding T cells, as described in Example 1. Blood is obtained from HLA-DRB1*-typed donors or patients. Peripheral blood mononuclear cells (PBMC) are isolated via centrifugation in Biocoll Separating Solution (Biochrom, Berlin, Germany) and either used directly after preparation or cryopreserved for later use. Cells are cultured in RPMI 1640 with L-Glutamine (Invitrogen, Karlsruhe, Germany), supplemented with 10% heat-inactivated, pooled human serum and 100 U/ml Penicillin-Streptomycin (Invitrogen, Karlsruhe, Germany). Aspergillus and Candida antigen-specific T cell lines are generated by incubation of 1×10⁷ whole PBMC per well in 6-well culture plates with the FHT peptide antigen for 7 days. Lymphocyte cultures are supplemented with 5 U/ml IL-2 (Proleukin, Chiron, Ratingen, Germany) every other day and culture medium replenished as needed. The T cell clones are generated by stimulating PBMCs repeatedly with 1 μg/ml FHT peptide once weekly for 4 weeks. Subsequently T cell clones are generated by limiting dilution in 96-well plates and expanded using the rapid expansion protocol as described by Beck et al⁵⁰. This small scale culture system may be scaled up and adapted to a “closed system” whereby clinical grade T cells suitable for infusion back into patients can be generated.

Suitable methods for selecting Aspergillus and Candida antigen-specific T cells include the use of a fluorescence-activated cell sorter (FACS). Following exposure of the donor or patient PBMCs to the HTYTIDWTKDAVTWS peptide, the responding T cells are labeled based on activation markers such as the upregulation of CD69 or by behavioural characteristics such as the secretion of IFN-γ. The labeling is achieved using an antibody specific for the activation marker or the secreted cytokine and such antibody is conjugated to a fluorochrome. The cells can then be separated and selected through a flow cytometer equipped for FACS analysis.

Alternatively, labeling of the responding T cells is based on the binding of an HLA multimer (HLA-DRB1*03 or HLA-DRB1*13), which is conjugated to a fluorescent marker, to the specific TCR on the surface of the Aspergillus and Candida antigen-specific T cell. Suitable methods for selecting T cells include the Cytokine Secretion Assay System which is manufactured by Miltenyi Biotec and involves four key steps: 1) exposure of PBMCs (which contain antigen-presenting cells) from a blood sample to an immunogenic antigen (such as the peptide HTYTIDWTKDAVTWS); 2) the responding Aspergillus and Candida antigen (eg HTYTIDWTKDAVTWS peptide)-specific T cells begin to secrete IFN-γ which is associated with an active and effective T cell immune response and these responding T cells are labelled with a bi-specific catch antibody which simultaneously binds to CD45 (a T cell marker) and IFN-γ; 3) a second antibody then labels the captured IFN-γ and in so doing labels the T cell which has secreted the IFN-γ this second antibody is also conjugated to a magnetic bead; 4) the cells are passed through a magnetic column and the responding T cells (which recognised the FHT Peptide as evidenced by the secretion of IFN-γ are retained in the column by the attached magnetic bead. The non-labelled cells are washed through and then the magnetic field is switched off and the labelled cells are released and collected as the positive fraction.

Methods for making and using peptide-loaded HLA multimers are described, for example, in Altman et al (1996) Science 274, 94-96; Kuabel et al (2002) Nature Medicine 8, 631-637; and Neudorfer et al (2007) J. Immunol. Methods 320, 119-131.

The purity of a T cell population may be assessed using the fluorescently labelled HLA multimer/peptide complex as discussed above.

Suitable methods for selecting T cells also include the MHC (HLA) Multimer System, available through Proimmune and Stage Pharmaceutical, which works by creating an artificial construction of Class II HLA molecules which bind, in the present case, the peptide present in the complexes of the invention (eg the HTYTIDWTKDAVTWS peptide). These soluble, standalone HLA molecules may be constructed in a multimeric configuration so that a single multimer has 4-5 HLA molecules each loaded with a peptide as defined as present in the complex of the invention, such as HTYTIDWTKDAVTWS peptide. These multimers may also be attached to a magnetic bead as above. The multimers are released into a blood sample, and the HLA:peptide complex will bind with T cell receptors that recognise the complex and hence will label the T cells which will recognise and mount an immune response against Aspergillus. The cell sample is passed through a magnetic column, and the labelled cells are retained and then released as described above.

The Aspergillus and Candida antigen-specific T cell is isolated for further use. With some techniques it is possible to isolate a sufficient number of specific T cells for therapeutic use directly, but it may be necessary to expand or clone the T cells to produce a sufficient number. For adoptive immunotherapy it is generally preferred to use a technique which allows for the isolation of sufficient numbers of cells directly since this can be achieved within a day (whereas cell expansion may take several weeks).

A suitable procedure for identifying pathogen-specific donor clones is described in Perruccio et al (2005) Blood 106, 4397-4406.

The Aspergillus and Candida antigen-specific T cells of the invention are useful in therapy. Thus, a second aspect of the invention provides Aspergillus and Candida antigen-specific T cells obtainable by the foregoing methods of the invention. Such T cells are able to recognize a complex of the first aspect of the invention.

A third aspect of the invention provides an Aspergillus and Candida antigen-specific T cell which is able to recognize the HLA-DRB1*03- or HLA-DRB*13-presented HTYTIDWTKDAVTWS peptide. Typically, this T cell is CD3⁺. It may be CD3⁺CD4⁺ or it may be CD3⁺CD8⁺. Typically, this T cell will produce cytokines and proliferate when in the presence of the HTYTIDWTKDAVTWS peptide when presented by HLA-DRB1*03 or HLA*DRB1*13, for example when presented by an antigen presenting cell which expresses HLA-DRB1*03 or HLA-DRB1*13. Preferably the T cell also recognizes HLA-DRB1*03 or HLA-DRB*13-presented HTYVIDWTKDAVTWS. The T cells in this aspect of the invention typically are not T cells which are able to detect the HLA-DRB1*04-presented peptide HTYTIDWTKDAVTWS.

The activated Aspergillus and Candida antigen-specific T cells of the invention may be packaged and presented for use as a medicament. The invention also includes a pharmaceutical preparation comprising Aspergillus and Candida antigen-specific T cells of the invention and a pharmaceutically acceptable carrier. Typically, the carrier is sterile and pyrogen free. The invention also includes the Aspergillus and Candida antigen-specific T cells packaged and presented for use in combating Aspergillus or Candida albicans infection. Preferably, the patients to be treated are ones which carry Class II HLA-DRB1*03 and/or HLA-DRB1*13.

For the treatment of patients following allogeneic HSCT, the Aspergillus and Candida antigen-specific T cells typically are isolated from the donor providing the stem cells. If the number of Aspergillus and Candida antigen-specific T cells is not sufficient through direct recovery of the cells from the donor, then the cells may be expanded through well established cell proliferation techniques.

However, in some embodiments the T cells are autologous (ie from the patient), and in some embodiments the T cells may be from an unrelated donor, for example from a bank of T cells which have been HLA-typed.

The activated Aspergillus and Candida antigen-specific T cells may be stored in any suitable way, for example by cryopreservation in medium containing serum and DMSO as is well known in the art.

The methods of the invention therefore include methods of adoptive immunotherapy.

It will be appreciated that the HLA multimers loaded with peptide (HLA-DRB1*03- or HLA-DRB1*13-peptide complexes of the invention) may be used to assess whether a blood sample contains Aspergillus and Candida antigen-specific T cells. This may be done in a sample from a patient before treatment and/or during the course of treatment or after treatment.

The Aspergillus and Candida antigen-specific T cells contain a T cell receptor (TCR) which is involved in recognising cells which express the polypeptide. It is useful if the cDNA encoding the TCR is cloned from the Aspergillus and Candida antigen-specific T cells and transferred into autologous or donor-derived T cells for expression, thereby obviating the need to find naturally occurring Aspergillus and Candida antigen-specific T cells expressing the TCR.

The TCRs of Aspergillus and Candida antigen-specific T cell clones of the invention specific for the complex of the first aspect of the invention are cloned. The TCR usage in the Aspergillus and Candida antigen-specific T cell clones is determined using (i) TCR variable region-specific monoclonal antibodies and (ii) RT-PCR with primers specific for Vα and Vβ gene families. A cDNA library is prepared from poly-A mRNA extracted from the Aspergillus and Candida antigen specific T cell clones. Primers specific for the C-terminal portion of the TCR α and β chains and for the N-terminal portion of the identified Vα and β segments are used. The complete cDNA for the TCR α and β chain is amplified with a high fidelity DNA polymerase and the amplified products cloned into a suitable cloning vector. The cloned α and β chain genes may be assembled into a single chain TCR by the method as described by Chung et al (1994) Proc. Natl. Acad. Sci. USA 91, 12654-12658. In this single chain construct the VαJ segment is followed by the VβDJ segment, followed by the Cβ segment followed by the transmembrane and cytoplasmic segment of the CD3 ξ chain. This single chain TCR is then inserted into a retroviral expression vector (a panel of vectors may be used based on their ability to infect mature human CD4⁺ T lymphocytes and to mediate gene expression: the retroviral vector system Kat is one preferred possibility (see Finer et al (1994) Blood 83, 43). High titre amphotrophic retrovirus are used to infect purified CD4⁺ T lymphocytes isolated from the peripheral blood of tumour patients following a protocol published by Roberts et al (1994) Blood 84, 2878-2889, incorporated herein by reference. Anti-CD3 antibodies are used to trigger proliferation of purified CD4⁺ T cells, which facilitates retroviral integration and stable expression of single chain TCRs. The efficiency of retroviral transduction is determined by staining of infected CD4⁺ T cells with antibodies specific for the single chain TCR. Patients may be treated with in between 10⁶ to 10⁸ (most likely 10⁷) autologous, transduced Aspergillus and Candida antigen-specific T cells.

Other suitable systems for introducing genes into T cells are described in Moritz et al (1994) Proc. Natl. Acad. Sci. USA 91, 4318-4322, incorporated herein by reference. Eshhar et al (1993) Proc. Natl. Acad. Sci. USA 90, 720-724 and Hwu et al (1993) J. Exp. Med. 178, 361-366 also describe the transfection of T cells.

Thus, a fourth aspect of the invention provides a TCR which recognizes a complex of the invention, in particular the peptide HTYTIDWTKDAVTWS complexed with a HLA-DRB1*03 or HLA-DRB1*13 molecule. Typically, the peptide is presented on a dendritic cell or monocyte which present the antigen to T cells. The T cells then secrete cytokines which in turn activate monocytes and neutrophils for enhanced killing of Aspergillus.

As well as the TCR, functionally equivalent molecules to the TCR are included in the invention. These include any molecule which is functionally equivalent to a TCR which can perform the same function as a TCR. In particular, such molecules include genetically engineered three-domain single-chain TCRs as made by the method described by Chung et al (1994) Proc. Natl. Acad. Sci. USA 91, 12654-12658, incorporated herein by reference, and referred to above.

Typically, the TCR or a functionally equivalent molecule to the TCR, recognizes a complex of the first aspect of the invention present on the surface of an antigen-presenting cell.

The invention also includes a polynucleotide encoding the TCR or functionally equivalent molecule, and an expression vector encoding the TCR or functionally equivalent molecule thereof. Expression vectors which are suitable for expressing the TCR of the invention include viral vectors such as retroviral vectors, lentiviral vectors, adenoviral vectors, vaccinia vectors (including the replication-deficient MVA strain). It is, however, preferred that the expression vectors are ones which are able to express the TCR in a T cell following transfection.

The invention also includes T cells, preferably CD4⁺ T cells, which have been transfected with a polynucleotide or expression vector which expressed the above mentioned TCR or functionally equivalent molecule. The T cells may be obtained from the patient or, in the case of an allogeneic HSCT patient, from a closely matched donor with respect to HLA type.

The invention also includes a method of combating Aspergillus or Candida albicans infection in a patient, or of combating allergy to Aspergillus or Candida albicans in a patient, the method comprising administering to the patient an effective number of Aspergillus and Candida antigen-specific T cells of the invention. For the avoidance of doubt, the Aspergillus and Candida antigen-specific T cells include those obtainable by the method for producing activated Aspergillus and Candida albicans antigen-specific T cells in vitro, described above, Aspergillus and Candida antigen-specific CD4⁺ T cells which are CD3⁺ and which recognize the complex of the invention, in particular the HTYTIDWTKDAVTWS complexed with a HLA-DRB1*03 or HLA-DRB1*13 molecule, and those prepared by transfection of a T cell with a polynucleotide or expression vector which expresses the above-mentioned TCR or functionally equivalent molecule. The T cells of the invention are able to combat Aspergillus or Candida albicans infection selectively by secreting cytokines in response to the antigen. Preferably, the patient is one who carries Class II HLA-DRB1*03 or Class II HLA-DRB1*13.

In respect of all treatments methods, the Aspergillus may be A. fumigatus, A. flavus, A. nidulans or A. terrea. Preferably, the Aspergillus is A. fumigatus.

The T cells may be autologous or they may be HLA matched. If the patient is undergoing allogeneic HSCT, typically the Aspergillus and Candida antigen-specific T cells are from the donor. When allergy to Aspergillus is to be combated in the patient, it is preferred that the T cells are T_{H}1 helper cells or T regulatory cells.

Thus, for patients who demonstrate an allergic reaction to Aspergillus antigens, the complexes of the invention may be used to select antigen-specific T regulatory cells which may suppress allergic responses. It will be appreciated the T regulatory cells can be identified by specific markers such as CD25, Foxp3, GITR, and CD127 and the co-expression of these markers with antigen-specificity for the peptide/HLA colpexes of the invention will allow the selection of T regulatory cells to modulate allergic responses to Aspergillus antigens. Thus, a fifth aspect of the invention provides a method of selecting T regulatory cells for the suppression of an allergic response to Aspergillus, the method comprising the use of the complexes of the invention in combination with markers for T regulatory cells in order to create a cellular formulation which can be used to suppress allergic reactions to Aspergillus. Conveniently, the cell formulation may be made using high levels of IL-2, and T regulatory cells can be selected as CD3⁺CD25⁺.

The effective clearance of pathogens from the human body sometimes depends on the appropriate T cell response. T_{H}1 responses are associated with cellular pathways involving cytotoxic and phagocytic effector functions while T_{H}2 responses typically involve the production of antibodies by B cells which are “helped” by T_{H}2 T cells. Clearance of Aspergillus infections is normally associated with T_{H}1 responses, and the ex vivo selection and expansion of antigen-specific T_{H}1 T cells using the peptides of the invention with subsequent infusion of the expanded T_{H}1 T cells into the patient may should facilitate the clearance of the Aspergillus infection by reshaping the dominant immune response in the patient.

A sixth aspect of the invention provides a method of combating Aspergillus or Candida albicans infection, the method comprising the steps of (1) obtaining T cells from the patient; (2) introducing into said cells a polynucleotide encoding a TCR, or a functionally equivalent molecule, as defined above; and (3) introducing the cells produced in step (2) into the patient. The transfected T cells are able to help fight off the Aspergillus, in particular A. fumigatus, or Candida albicans. Preferably, the patients to be treated carry Class II HLA-DRB1*03 and/or Class II HLA-DRB1*13.

A seventh aspect of the invention provides a method of combating Aspergillus or Candida albicans infection in a patient which patient carries Class II HLA-DRB1*03 and/or Class II HLA-DRB1*13, the method comprising the steps of (1) obtaining dendritic cells or other antigen-presenting cells from said patient; (2) contacting said dendritic or other antigen-presenting cells ex vivo with a peptide comprising the amino acid sequence HTYTIDWTKDAVTWS or a portion thereof, or a variant thereof wherein the side chains of one or two or three or four or five or six or seven of the amino acid residues are altered, wherein the peptide comprising the portion, or variant, is capable of binding HLA-DRB1*03 and/or HLA-DRB1*13; and (3) reintroducing the so treated dendritic or other antigen-presenting cells into the patient. Preferably, the patients to be treated carry Class II HLA-DRB1*03 or Class II HLA-DRB1*13. Typically, the HLA-status of the patient is determined prior to treatment and patients are selected who carry HLA-DRB1*03 or HLA-DRB1*13.

The methods of combating Aspergillus or Candida albicans infection, and the pharmaceutical compositions and medicaments, of the invention may be combined with other anti-fungal treatments, such as the use of voriconazole.

Particularly preferred groups of patients to treat include (a) patients who are immunosuppressed following allogeneic HSCT, who may be treated with Aspergillus and Candida antigen-specific T cells of the invention selected from the donor of the HSCs; (b) patients who are immunosuppressed due to chemotherapy, HIV infection or by the use of immunosuppressive drugs for autoimmune disease, who may be treated with Aspergillus and Candida antigen-specific T cells of the invention selected from the patient (because these are likely to be present in very small numbers, the cell numbers may be expanded on an ex vivo basis; (c) allergy patients in which antigen-specific regulatory T cells are used or T_{H}1 cells are infused to re-direct an T_{H}2 response. In all cases it is preferred that the patient and the donor have the HLA-DRB1*03 or HLA-DRB1*13 allele.

The peptide HTYTIDWTKDAVTWS may be used to create a monoclonal or polyclonal antibody either on a patient-specific basis or batch manufacturing basis wherein the antibody is used to prevent or treat infection by Aspergillus or Candida albicans or to induce a primary or secondary humoral or cellular immune response to Aspergillus or Candida albicans in a patient. The antibody will include idiomatic derivations of antibodies specific for the peptide HTYTIDWTKDAVTWS. Preferably, the antibody recognises specifically the peptide HTYTIDWTKDAVTWS when presented by a Class II HLA-DRB1*03 or Class II HLA-DRB1*04 or Class II HLA-DRB1*13 molecule. The antibody does not bind to the peptide FHTYTIDWTKDAVTW.

Thus, an eighth aspect of the invention provides a method of preparing an antibody which recognises the peptide HTYTIDWTKDAVTWS. The method may involve immunisation of an animal such as rabbit or mouse or horse or camel. The method may use hybridoma technology to produce monoclonal antibodies. The method may use in vitro selection techniques such as phage display. Preferably, the peptide is presented by a Class II HLA-DRB1*03 or a Class II HLA-DRB1*04 or a Class II HLA-DRB1*13 molecule.

The antibody may be packaged and presented for use in medicine, particularly for use in combating Aspergillus or Candida albicans infection. The invention also provides a method of combating Aspergillus or Candida albicans infection in a patient by administering an effective amount of the antibody to the patient. Preferably, the patient is one who carries Class II HLA-DRB1*03 or Class II HLA-DRB1*04 or Class II HLA-DRB1*13.

The antibody may be monoclonal or polyclonal. Methods of preparing antibodies for administration, including pharmaceutical compositions, are known.

The Aspergillus and Candida antigen-specific TCR molecules of the invention may be used within a diagnostic strategy to determine whether a patient may be infected by Aspergillus as evidenced by the labelling of Aspergillus and Candida antigen-specific T cells with soluble standalone Aspergillus and Candida antigen-specific TCR molecules attached to an appropriate labelling system (typically an antibody attached to a fluorochrome). Thus, the invention includes a method of determining whether an individual is infected with Aspergillus, the method comprising using a TCR molecule of the invention. The soluble TCR molecules are, in some respects, similar to monoclonal antibodies and can be used to quantify antigen-presenting cells which are presenting a peptide of the invention.

Suitable samples from the patient include a sample following bronchoscopic examination, such as one obtained following lung lavage.

The patients treated by the therapeutic methods of the invention are preferably human patients. The patients treated by the therapeutic methods of the invention preferably are HLA-DRB1*03 or HLA-DRB1*13. Thus, it may be convenient to determine whether an individual to be treated is HLA-DRB1*03 or HLA-DRB1*13 or generally determine the HLA genotype. It is also preferred that the donor of any T cell for use in the therapeutic methods of the invention is HLA-DRB1*03 or HLA-DRB1*13.

As mentioned above, it has surprisingly been found that a peptide epitope (and related peptides) of the Aspergillus fumigatus Crf1 protein not only gives rise to protection against Aspergillus, but also gives rise to protection against the unrelated yeast Candida albicans.

Thus, a ninth aspect of the invention vaccine against Candida albicans infection comprising

(a) a peptide comprising the amino acid sequence HTYTIDWTKDAVTWS or a portion thereof, or a variant thereof wherein the side chains of one or two or three or four or five or six or seven of the amino acid residues are altered, wherein the peptide comprising the portion, or variant, is capable of binding HLA-DRB1*03 and/or HLA-DRB1*04 and/or HLA-DRB1*13, or
(b) a polypeptide which is a fusion of any of the peptides of (a) and another peptide, or
(c) a polynucleotide encoding a peptide of (a) or a polypeptide of (b) or
(d) an expression vector capable of expressing a peptide of (a) or a polypeptide of (b).

Binding to HLA molecules can be assessed as described above in relation to the first aspect of the invention.

It will be appreciated that in this aspect of the invention, and the other aspects of the invention directed at the treatment of Candida albicans, the peptide may be presented by any suitable HLA molecule.

The peptides in the vaccine of the ninth aspect of the invention are less than 10 000 in molecular weight, preferably less than 8 000, more preferably less than 5 000 and typically about 4 000 or 3 000 or 2 000. In terms of the number of amino acid residues, the peptides of the invention may have fewer than 100 residues, preferably fewer than 80 residues, more preferably fewer than 50 residues or about 40 or 30 or 20 or 19 or 18 or 17 or 16 or 15 or 14 or 13 or 12 or 11.

A peptide or a polypeptide or a polynucleotide or an expression vector as defined in the vaccine of the ninth aspect of the invention may be used for combating Candida albicans infection or for combating allergy to Candida albicans. The invention also includes a method of combating Candida albicans infection in a patient or a method of combating allergy to Candida albicans, the method comprising administering to the patient an effective amount of a peptide or a polypeptide or a polynucleotide or an expression vector as defined in the vaccine of the ninth aspect of the invention wherein the amount of said peptide or polypeptide or amount of said polynucleotide or amount of said expression vector is effective to provoke an anti-Candida albicans response in said patient.

It will be appreciated that the peptides or polypeptides of the vaccine of the ninth aspect of the invention may be presented by an antigen presenting cell, such as a dendritic cell, and that this antigen presenting cell, when presenting the peptide or polypeptide, may be used as a vaccine against Candida albicans infection, and may be used to combat Candida albicans infection. Thus, the invention also includes a method for combating Candida albicans infection in a patient, the method comprising administering to the patient an effective amount of an antigen presenting cell which is presenting the peptide or polypeptide as defined in the vaccine of the ninth aspect of the invention.

It will be appreciated that the peptides as defined in the vaccine of the ninth aspect of the invention may be used to identify, isolate or select Aspergillus and Candida antigen-specific T cells by the methods described above. These T cells are useful in medicine, for example for combating Candida albicans infection or for combating allergy to Candida albicans. Thus, the invention includes a method of combating Candida albicans infection in a patient or of combating allergy to Candida albicans in a patient the method comprising administering to the patient an effective number of Aspergillus and Candida antigen-specific T cells which are able to recognise the HLA-presented peptide HTYTIDWTKDAVTWS, or population of T cells which has been activated using the peptide or polypeptide as defined in the vaccine of the ninth aspect of the invention. The peptide HTYTIDWTKDAVTWS may be presented by HLA-DRB1*03, or HLA-DR1*04 or HLA-DRB1*13. The invention also includes a pharmaceutical composition comprising the T cells as defined and a pharmaceutically acceptable carrier.

The preferences and suitabilities for isolating T cells for use in combating Candida albicans are essentially as described above except that HLA-DR1*04 may be used to present the peptides as well as HLA-DR1*03 and HLA-DR1*13.

It will be appreciated that T cell receptors, and polynucleotides encoding the T cell receptors, may be isolated from the T cells by methods as described above, and that these T cell receptors and polynucleotides are useful in medicine. In particular, the invention includes a method of combating Candida albicans infection in a patient, the method comprising the steps of (1) obtaining T cells from the patient; (2) introducing into said cells a polynucleotide encoding a T cell receptor (TCR), or a functionally equivalent molecule, obtainable from an Aspergillus and Candida antigen-specific T cell which is able to recognise the HLA-presented peptide HTYTIDWTKDAVTWS; (3) introducing the cells produced in step (2) into the patient. The invention also includes a method of combating Candida albicans infection in a patient, the method comprising the steps of (1) obtaining antigen presenting cells, typically dendritic cells from said patient; (2) exposing said antigen presenting cells ex vivo with a peptide or a polypeptide or a polynucleotide or expression vector as defined in the vaccine of the ninth aspect of the invention; and (3) reintroducing the so treated cells into the patient.

The invention also includes a method of determining whether an individual is infected with Candida albicans, the method comprising determining whether an individual contains Aspergillus and Candida antigen-specific T cells which are able to recognise the HLA-presented peptide HTYTIDWTKDAVTWS. The invention also includes a method of determining whether an individual is infected with Candida albicans, the method comprising using a TCR obtainable from the Aspergillus and Candida antigen-specific T cells as disclosed above.

It will be appreciated that a further step of detecting Candida albicans, or a specific part thereof, may be employed to confirm infection. For example, a Candida albicans-specific PCR detection system or a Candida albicans-selective immunoassay may be employed, which uses an antibody.

The invention also gives rise to further peptides that may be used in medicine, and to obtain further useful molecules for medicine.

Thus, a tenth aspect of the invention provides a peptide of less than 10 000 molecular weight comprising the amino acid sequence HTYTIDWTKDAVTWS or a portion thereof, or a variant thereof wherein the side chains of one or two or three or four or five or six or seven of the amino acid residues are altered, wherein the peptide comprising the portion, or variant, is capable of binding HLA-DRB1*03 and/or HLA-DRB1*13, provided that the peptide is not a peptide of less than 10 000 molecular weight comprising the amino acid sequence FHTYTIDWTKDAVTW or a portion thereof, or a variant thereof wherein the side chains of one or two or three or four or five or six or seven of the amino acid residues are altered, wherein the peptide comprising the portion, or variant, is capable of binding HLA-DRB1*04.

Methods of determining binding to Class II HLA molecules are described above. The preferred sizes and nature of the peptides, and the preferred numbers of alterations to the amino acid sequence and length of portion are as described above. The peptide is preferably one which, when bound to HLA-DRB1*03 or HLA-DRB1*13 is capable of identifying Aspergillus and Candida antigen-specific T cells as disclosed above. It is particularly preferred if the peptides bind selectively to HLA-DRB1*03 or HLA-DRB1*13.

A particularly preferred peptide is HTYTIDWTKDAVTWS. The peptide per se do not include FHTYTIDWTKDAVTW, or PVATPQETFHTYTID, or PQETFHTYTIDWTKD, or TIDWTKDAVTWSIDG, or TKDAVTWSIDGAWR, or HTYTIDWTKDA. It will be appreciated from the following that in some applications the peptides of the tenth aspect of the invention may be used directly (le they are not produced by expression of a polynucleotide in a patient's cell or in a cell given to a patient); in such applications it is preferred that the peptide has fewer than 50 residues, preferably fewer than 45 residues or about 40 or 30 or 25 or 24 or 23 or 22 or 21 or 20 or 19 or 18 or 17 or 16 or 15 or 14 or 13 or 12 or 11 residues.

An eleventh aspect of the invention provides a polypeptide which is a fusion of a peptide of the tenth aspect of the invention and another peptide. The other peptide may be fused to the N-terminus of the peptide of the invention or to the C-terminus of the peptide of the invention or, in some embodiments other peptides (either the same or different) may be fused to both the N-terminus or C-terminus of the peptide of the invention. Typically, these peptides may be useful in ensuring the peptide of the tenth aspect of the invention is directed through the endosomal pathway for HLA Class II presentation. Alternatively, these fused peptides may be linked to known immunogenic substances such that the combined peptide structure is more effective in inducing an effective human immune response against Aspergillus or Candida albicans.

The peptide of the tenth aspect of the invention may be comprised within, or fused to, an HLA molecule such that the peptide can occupy the peptide-binding groove of the HLA molecule. Fusion molecules of this type, although using a different peptide, have been synthesised by Mottez et al (1995) J. Exp. Med. 181, 493-502, incorporated herein by reference. Preferably, the HLA molecule to which the peptide of the invention is fused is HLA-DRB1*03 or HLA-DRB1*04.

The peptide of the invention may also be fused to another peptide comprising an HLA epitope.

Typically, the fusion polypeptide contains all or 11 or 12 or 13 or 14 contiguous amino acids of the peptide HTYTIDWTKDAVTWS; the 12 or 13 or 14 contiguous amino acids may contain one or two or three or four or five or six or seven replacements typically of conservative amino acids. Typically, the fusion polypeptide has a molecular weight of less than 10 000 or 8 000 or 5000 or 4000 or 3000 or 2000. Typically the fusion polypeptide has from around 20 to 100 amino acids such as 20 to 50, or 25 to 40.

A preferred fusion polypeptide of the invention is between a peptide of the invention and the HLA Class II invariant chain which stabilises the HLA Class II molecule and which is involved in directing the HLA molecule through the endosomal pathway leading to expression on the surface of a cell. It will be appreciated that the polypeptide is typically used in an HLA-DRB1*03 or HLA-DRB1*13-specific context.

A twelfth aspect of the invention provides a polynucleotide encoding a peptide as defined in the tenth aspect of the invention or a polypeptide as defined in the eleventh aspect of the invention. The polynucleotide may be DNA or RNA and it may or may not contain introns so long as it codes for the peptide. Of course, it is only peptides which contain naturally occurring amino acid residues joined by naturally-occurring peptide bonds which are encodable by a polynucleotide. It will be appreciated that the polynucleotide is typically used in an HLA-DRB1*03 or HLA-DRB1*13-specific context.

A thirteenth aspect of the invention provides an expression vector capable of expressing a peptide according to the tenth aspect of the invention or a polypeptide according to the eleventh aspect of the invention. It will be appreciated that the expression vector is typically used in an HLA-DRB1*03 or HLA-DRB1*13-specific context.

Methods for manipulating, changing and cloning nucleic acid molecules are well known in the art, for example Sambrook J and Russell, D W, Molecular Cloning, A Laboratory Manual, 3^{rd} Edition, 2001, Cold Spring Harbor Laboratory Press describes such techniques including PCR methods. Suitable expression vectors include viral based vectors such as retroviral or adenoviral or vaccinia virus vectors or lentiviral vectors or replication deficient MV vectors. Suitable general cloning vectors include plasmids, bacteriophages (including λ and filamentous bacteriophage), phagemids and cosmids. Suitable host cells include bacteria, yeast, insect and mammalian cells. Particular bacteria include Escherichia coli, Bacillus subtilis and Salmonella typhimurium. Particular yeast cells include Saccharomyces cerevisiae and Schizosaccharomyces pombe. Particular mammalian cells include CHO cells, COS cells and other mammalian cells such as antigen presenting cells.

It will be appreciated that certain host cells of the invention are useful in the preparation of the peptides or polypeptides of the invention including the TCR molecules of the invention, for example bacterial, yeast, mammalian and insect cells. Thus, a further aspect of the invention provides a host cell comprising a polynucleotide of the invention or an expression vector of the invention. It is particularly preferred that the host cell is a human host cell, such as an antigen presenting cell, which carries Class II HLA-DRB1*04.

A further aspect of the invention provides a method of producing a peptide of the tenth aspect of the invention or a polypeptide of the eleventh aspect of the invention, the method comprising culturing host cells which contain a polynucleotide or expression vector which encodes the peptide or polypeptide and obtaining the peptide or polypeptide from the host cell or culture medium. The peptides and polypeptides of the invention may also be chemically synthesised as discussed above.

The peptides of the tenth aspect of the invention may be used in the production of Aspergillus and Candida antigen-specific T cells, particularly CD4⁺ T cells, and in particular for a peptide which has the amino acid sequence HTYTIDWTKDAVTWS or a portion thereof, for example a portion with from 11 or 12 or 13 or 14 contiguous amino acids, and which is be presented by HLA-DRB1*03 or HLA-DRB1*13.

Thus, further aspects of the invention provide compositions which comprise a peptide according to the tenth aspect of the invention or a polypeptide according to the eleventh aspect of the invention which compositions are suitable for raising T cell clones, particularly CD4⁺ T cell clones, specific for a peptide which has the amino acid sequence HTYTIDWTKDAVTWS or a portion thereof, for example a portion with from 11 or 12 or 13 or 14 contiguous amino acids, and which is be presented by HLA-DRB1*03 or HLA-DRB1*13. Such compositions are typically sterile and pyrogen free. The composition is typically a pharmaceutical composition which contains a pharmaceutically acceptable carrier. Typically, for the generation of T cells, particularly CD4⁺ T cells, the peptides are used in the range 200 μM to 1 nM. Typically, the composition is an aqueous composition. In some instances it may be desirable to include a peptide-solubilising agent such as DMSO in the composition.

It will be appreciated that the peptides, polypeptides, polynucleotide, expression vectors, T cell receptors and antigen-presenting cells of the invention may all be provided in a pharmaceutical composition containing a pharmaceutically acceptable carrier. Typically, these compositions would be sterile and pyrogen free.

The peptides, polypeptides, polynucleotides and expression vectors of the invention may be packaged and presented for use as a medicament. In particular, they are of use in combating Aspergillus or Candida albicans infection for example they may be used in a vaccine. By “combating Aspergillus infection” in all relevant aspects of the invention we include treating patients who have an Aspergillus infection, for example patients who have ABPA or Invasive Aspergillosis (IA) or Aspergilloma or Chronic Aspergillus sinusitis. By “combating Candida albicans infection” in all relevant aspects of the invention we include treating patients who have candidiasis such as human diseases associated with Candida albicans include cutaneous candidiasis syndromes, chronic mucocutaneous candidiasis, gastrointestinal tract candidiasis, genitourinary tract candidiasis, respiratory tract candidiasis, hepatosplenic candidiasis and systemic candidiasis. Of particular note are post-transplant candidiasis, HIV-associated candidiasis and abdominal surgery associated candidiasis.

We also include administering the peptides, polypeptides, polynucleotides and expression vectors (either alone or in combination with, or present in or on, a suitable HLA matched antigen presenting cell such as a dendritic cell or B cell or monocytes or a synthetic APC) to not only patients who have an Aspergillus or Candida albicans infection, but also to those at risk of Aspergillus or Candida albicans infection. Patients at risk of Aspergillus or Candida albicans infection include those who are immunocompromised or immunodepleted such as those undergoing allogeneic HSCT, organ transplant patients, autoimmune patients receiving immunosuppressive drugs, patients with genetic immune disorders, AIDS patients, or patients undergoing chemotherapy for cancer or leukaemia patients. Thus, it will be appreciated that “combating” includes preventing (or helping to prevent) Aspergillus or Candida albicans infection and treating a patient prophylactically.

Allergy to Aspergillus, in particular to A. fumigatus, and to Candida albicans are medical problems. The invention also includes the use of the peptides, polypeptides, polynucleotides and expression vectors of the invention for combating Aspergillus or Candida albicans allergy, particularly allergy to A. fumigatus. By “combating allergy to Aspergillus or Candida albicans” we include treating allergy or preventing allergy. The peptides of the invention are particularly useful in this regard.

In respect of any relevant aspect of the invention, the Aspergillus infection may be an infection with any Aspergillus spp, and in particular an Aspergillus spp or strain which includes the Crf1 protein in the fungal proteome, and more particularly includes the peptides of the invention, particularly the peptide HTYTIDWTKDAVTWS as a part thereof. Typically, the patient has or is at risk of obtaining an infection of Aspergillus fumigatus or Candida albicans. In respect of the methods making use of the peptides of the tenth aspect of the invention or T cells reactive thereto, preferably, the patient is HLA-DRB1*03- or HLA-DRB1*13-positive.

The invention also includes a method of combating Aspergillus or Candida albicans infection in a patient, the method comprising administering to the patient an effective amount of a peptide of the tenth aspect of the invention or polypeptide or polynucleotide or expression vector of the invention (either alone or in combination with, or present in or on, a suitable antigen presenting cell such as a dendritic cell or B cell or T cell or monocyte) wherein the amount of the peptide or polypeptide or polynucleotide or expression vector is effective to provoke an anti-Aspergillus or anti-Candida albicans response in said patient. Preferably, the patient is HLA-DRB1*03- or HLA-DRB1*13-positive. Preferably, the antigen-presenting cell is HLA-DRB1*03- or HLA-DRB1*13-positive. The efficacy of this method of using the invention can typically be measured by testing for the presence of Aspergillus and Candida antigen-specific T cells in a patient who otherwise is not able to generate an immune response.

In respect of any relevant aspect of the invention, the anti-Aspergillus or anti-Candida albicans response may be measured by Elispot, intracellular cytokine staining or by a proliferation assay, or by using HLA multimer technology.

A still further aspect of the invention includes a kit of parts comprising a peptide according to the invention or a polypeptide according to the invention or a polynucleotide according to the invention or an expression vector according to the invention and an antigen presenting cell which is HLA-DRB1*03 or HLA-DRB1*13 positive. The kit of parts is useful in the preparation of activated Aspergillus and Candida albicans specific antigens T cells as described below.

A yet still further aspect of the invention includes an antigen-presenting cell wherein its HLA Class II molecules are loaded with a peptide of the tenth aspect of the invention. Typically, in all relevant embodiments of the invention the antigen-presenting cell contains an HLA-DRB1*03 or HLA-DRB1*13 molecule which presents the peptide.

Preferred antigen-presenting cells in all relevant aspects of the invention are dendritic cells or B cells or monocytes derived from the donor in the case of the treatment of patients undergoing allogeneic HSCT, and for other treatments the antigen-presenting cells are preferably autologous dendritic cells or B cells or T cells (ie derived from the patient to be treated).

The antigen presenting cells may be loaded with the peptide in vitro as described in detail above. Alternatively, the antigen presenting cell may be a recombinant cell which expresses the peptide or polypeptide from a suitable polynucleotide or expression vector.

The antigen presenting cell, such as a dendritic cell, a B cell or a monocyte which is presenting the peptide or polypeptide may be used as a vaccine and may be packaged and presented for use as a medicament. The antigen presenting cell which is presenting the peptide may be prepared as a pharmaceutical composition (ie by preparing it in a pharmaceutically acceptable carrier) and it may also be packaged and presented for use in combating Aspergillus or Candida albicans infection in a patient. Preferably, the antigen presenting cell is autologous, or is compatible with the patient to whom it is to be administered based upon a close HLA match.

The invention also includes a method for combating Aspergillus or Candida albicans infection in a patient, the method comprising administering to the patient an effective amount of an antigen presenting cell which is loaded with the peptide as defined in the tenth aspect of the invention wherein the amount of said antigen presenting cell is effective to provoke an anti-Aspergillus or anti-Candida albicans response in said patient. Preferably, the patient is one who carries Class II HLA-DRB1*03 or HLA-DRB1*13. Preferably the antigen presenting cell is one which carries Class II HLA-DRB1*03 or HLA-DRB1*13, and presents the peptide. The antigen presenting cells (of any relevant aspect of the invention) may be used in cell doses from 10² to 10⁷ cells per kg of the patient's weight, depending on whether the cells are autologous or only HLA matched. In this and other relevant aspects of the invention, antigen presenting cells may be infused to switch on T cells in vivo.

T cells are switched on by antigen-presenting cells such as dendritic cells. Hence cell therapy can involve T cells that have been switched on ex vivo and then infused into the patient.

A further aspect of the invention provides a method for selecting Aspergillus and Candida antigen-specific T cells, the method comprising contacting a population of T cells with a peptide or polypeptide of the invention presented in a Class II HLA-DRB1*03 or HLA-DRB1*13 molecule to which said peptide binds.

Preferably, in all relevant aspects of the invention, the population of T cells are from an individual who has been exposed to Aspergillus, in particular A. fumigatus or exposed to Candida albicans.

The peptide of the tenth aspect of the invention may be used to generate an expansion of Aspergillus and Candida antigen-specific T cells for patients who are HLA-DRB1*03 or HLA-DRB1*13 positive. T cell receptors may also be obtained from the T cells. The T cells and TCRs are obtained using essentially the same methodology as described above in respect of other relevant aspects of the invention. An effective population of the T cells may be administered in order to combat Aspergillus or Candida albicans infection in a patient.

The invention also includes a method of combating Aspergillus or Candida albicans infection in a patient, the method comprising the steps of (1) obtaining T cells from the patient; (2) introducing into said cells a polynucleotide encoding the T cell receptor (TCR) as discussed, or a functionally equivalent molecule, (3) introducing the cells produced in step (2) into the patient.

The invention also includes a method of combating Aspergillus or Candida albicans infection in a patient, the method comprising the steps of (1) obtaining antigen presenting cells, typically dendritic cells from said patient; (2) exposing said antigen presenting cells ex vivo with a peptide according to the tenth aspect of the invention or a fusion polypeptide or with a polynucleotide encoding the same; and (3) reintroducing the so treated cells into the patient.

The invention also includes a method of determining whether an individual is infected with Aspergillus or Candida albicans, the method comprising determining whether an individual contains Aspergillus and Candida antigen-specific T cells as defined above and a method of determining whether an individual is infected with Aspergillus or Candida, the method comprising using a TCR as defined above.

The knowledge that the peptide of the tenth aspect of the invention binds the HLA-DRB1*03 or HLA-DRB1*13 molecule and is only recognised by responder T cells in this context means that the creation of HLA multimers that will directly bind the T cells can occur. This reagent can be used to directly select the Aspergillus and Candida antigen-specific T cells from the bulk culture in order to infuse cells with a high purity into a patient.

The invention also includes use of the HLA multimer (including the complexes of the invention) in conjunction with additional multimers of other specificities (to be defined) to fully characterise the product prior to infusion to ensure purity and safety of the product. Following infusion of Aspergillus and Candida antigen-specific T cells to the patient the reconstitution of Aspergillus or Candida albicans specific immunity could be monitored in the patient using the HTYTIDWTKDAVTWS/HLA-DRB1*03 or HLA-DRB1*13 or HLA-DRB1*04 (as the case may be) multimer directly ex viva

An additional application of this technology would be that instead of relying on the innate antigen presentation ability of the patient's own cells to present the peptide of the tenth aspect of the invention in order to expand antigen presenting cells, an artificial antigen presenting cell could be used which consists of either cell lines deficient in all HLA but HLA-DRB1*03 and HLA-DRB1*13 and HLA-DRB1*04 pulsed with the peptide or artificial antigen presenting cells which can are engineered to supply the co-stimulation required for T cell expansion along with the HLA-DRB1*03 or HLA-DRB1*13 peptide combination in order to stimulate and expand the Aspergillus and Candida antigen-specific T cells.

It will be appreciated from the above that the invention includes a complex comprising a Class II HLA-DRB1*03 or Class II HLA-DRB1*13 molecule bound to a peptide according to the tenth aspect of the invention. Conveniently, the complex is a soluble complex and is not cell-bound. Preferably, the Class II molecule is a MHC multimer, such as those discussed above. Preferably, the peptide in the complex is the HTYTIDWTKDAVTWS peptide, but may be any other peptide of the tenth aspect of the invention that will form a complex, and be useful in eliciting an anti-Aspergillus or anti-Candida albicans T cell response.

The peptides of the tenth aspect of the invention, alone or in combination with antigens from other pathogens, and the complexes of the first aspect of the invention may be used to activate immune cells within a blood or tissue sample or a cellular derivative thereof obtained from the patient or a donor without significant further selection or purification of cell types (an “Unselected Cell Formulation”) with a view to infusing the Unselected Cell Formulation in a patient in order to treat or prevent infection by Aspergillus or Candida albicans whether on a targeted basis or as one of several pathogens which may cause infection in a patient.

Typically, the Unselected Cell Formulation is characterised using the peptide of the tenth aspect invention complexed with a soluble MHC complex (HLA-DRB1*03 or HLA-DRB1*13) or using the complexes of the first aspect of the invention.

The peptides of the tenth aspect of the invention may be used within a diagnostic strategy to determine whether a patient may be infected by Aspergillus or Candida albicans as evidenced by the patient's immune system demonstrating a diagnostically relevant response to the peptides of the tenth aspect of the invention, typically when presented by HLA-DRB1*03 or HLA-DRB1*13, or to determine whether a patient may be vulnerable to infection by Aspergillus or Candida albicans by a defective or insufficient response to the peptides of the tenth aspect of the invention in particular the HTYTIDWTKDAVTWS peptide. The diagnostic applications include the use of the peptides of the tenth aspect of the invention, typically when presented by HLA-DRB1*03 or HLA-DRB1*04 or HLA-DRB1*13, or the use of complexes of the first aspect of the invention to identify suitable donors for allogeneic haematopoietic stem cell transplants wherein the objective is to have a donor which demonstrates immunity to Aspergillus or Candida albicans. The number of responder cells may be very low, so a small scale cell sort may be required to determine this. Thus, the invention includes a method of determining whether an individual is infected with Aspergillus or Candida albicans, the method comprising determining whether an individual contains any Aspergillus and Candida antigen-specific T cells, which T cells are able to bind and be activated by a Class II HLA molecule, in particular HLA-DRB1*03 or HLA-DRB1*13 presenting a peptide of the tenth aspect of the invention or which bind to the complex of the first aspect of the invention. Such T cells can be identified and quantified, for example using the T cell selection systems discussed above. Individuals who contain the Aspergillus and Candida antigen-specific T cells are likely to be combating an infection, whereas individuals who do not contain, or contain only a low level, may benefit from adoptive immunotherapy according to the invention. On the other hand, individuals who are identified as containing the Aspergillus and Candida albicans antigen-specific T cells may be useful as donors.

The terms “HLA” and “MHC” are used interchangeably herein.

The invention will now be described in more detail by reference to the following non-limiting examples and figures.

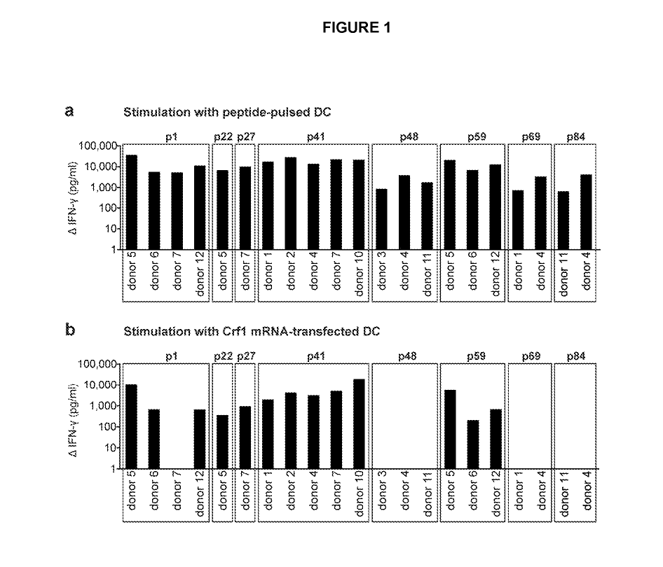

FIG. 1:

Response of peptide-specific T-cell clones to endogenously processed antigen that is presented by dendritic cells. (a) T-cell clones from different donors specific for the peptides p1, p22, p27, p41, p48, p59, p69 and p84 were stimulated with peptide-pulsed DC or (b) DC transfected with Crf1 mRNA. IFN-γ values are given after subtraction of the negative control. Number of T-cell clones reacting to Crf1 mRNA-transfected DC/number of peptide-specific T-cell clones: p1 donor 5 3/3, p1 donor 6 1/5, p1 donor 7 0/6, p1 donor 12 10/10, p22 donor 5 1/2, p27 donor 7 1/3, p41 donor 1 11/11, p41 donor 2 16/16, p41 donor 4 2/2, p41 donor 7 4/4, p41 donor 10 3/3, p48 donor 3 0/7, p48 donor 4 0/4, p48 donor 11 0/5, p59 donor 5 1/3, p59 donor 6 2/2, p59 donor 12 1/7, p69 donor 1 0/3, p69 donor 4 0/2, p84 donor 11 0/5, p84 donor 4 0/4. Shown is 1 clone per antigen and donor (representative experiments, n≧3).

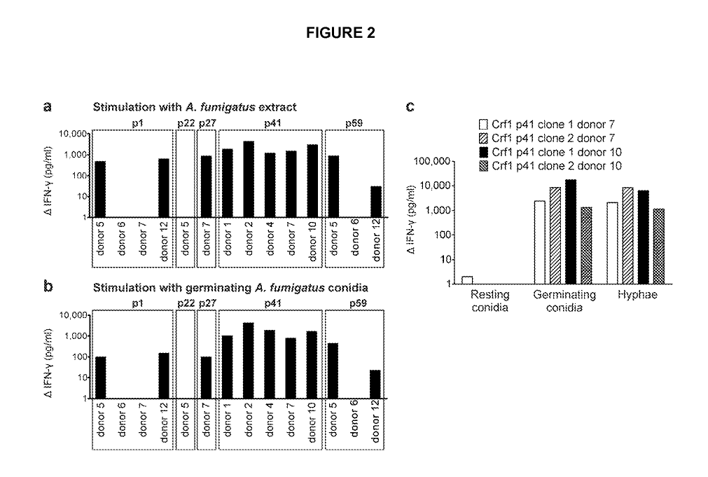

FIG. 2:

Response of Crf1 peptide-specific T-cell clones to stimulation with native A. fumigatus antigen. (a) T-cell clones from different donors specific for p1, p22, p27, p41 and p59 that responded to stimulation with Crf1 mRNA-transfected DC were stimulated with A. fumigatus cell extract or (b) inactivated germinating A. fumigatus conidia. IFN-γ values are given after subtraction of the negative control. Shown is 1 clone per antigen and donor (representative experiments, n≧3). (c) Crf1 p41-specific T-cell clones respond to stimulation with DC incubated overnight with germinating A. fumigatus conidia or hyphae but not to resting conidia. T cell clones were stimulated with A. fumigatus inactivated 0 h (resting conidia), 6 h (germinating conidia) or 12 h (hyphae) after incubation in growth medium.

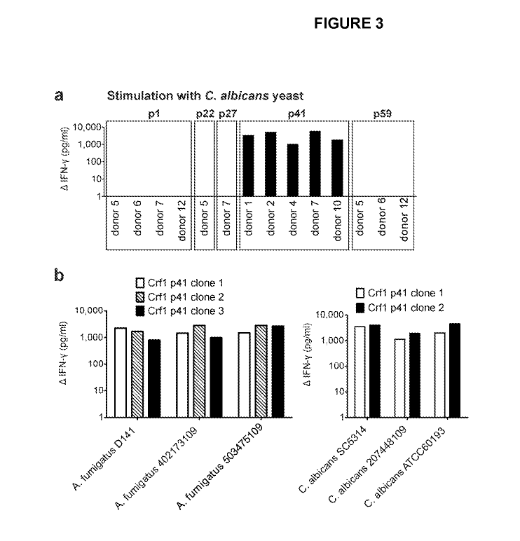

FIG. 3:

A. fumigatus Crf1 p41-specific T-cell clones are cross-reactive to C. albicans and can be activated by different clinical isolates of both fungi. (a) All A. fumigatus Crf1 p41-specific T-cell clones from different donors respond to stimulation with inactivated C. albicans yeast. IFN-γ values are given after subtraction of the negative control. Shown is 1 clone per antigen and donor (representative experiments, n≧3). (b) A. fumigatus Crf1 p41-specific T-cell clones respond to different inactivated clinical isolates of germinating A. fumigatus conidia and C. albicans yeast.

FIG. 4:

Cross-reactive Crf1 p41-specific T-cells show a diverse T-cell receptor (TCR) Vβ repertoire, multiple MHC class II restriction and high proliferative capacity. (a) The TCR Vβ repertoire of p41-specific CD4⁺ T-cell clones is divers in different donors. TCR chains were determined by flowcytometry, PCR and/or nucleotide sequence analysis. *TCRs differ on the level of nucleotide sequence. Nomenclature is according to Wei et al⁵³. (b) The Crf1 p41 epitope can be presented by HLA-DRB1*03, 04 and 13 and allows cross-recognition between HLA-DRB1*03 and 13. The HLA restriction of Crf1 p41-specific T-cell clones from 5 different donors was determined by stimulation with peptide-pulsed unmatched or partially matched LCL. IFN-γ values are given after subtraction of the negative control. (c) Crf1 p41-specific T-cells are not terminally differentiated and can be rapidly expanded in vitro. 10⁷PBMC could be expanded to a median of 3.5×10⁷ cells (range 3−4.9×10⁷ cells) within 14 days and the frequency of antigen-specific cells as determined by a HLA-DRB1*04 restricted tetramer reached levels of 42-63% (median 50.6%). T-cells were restimulated with autologous peptide-pulsed monocytes on day 7 and culture medium was supplemented with IL-2, IL-7 and IL-15 (representative experiments, n≧3).

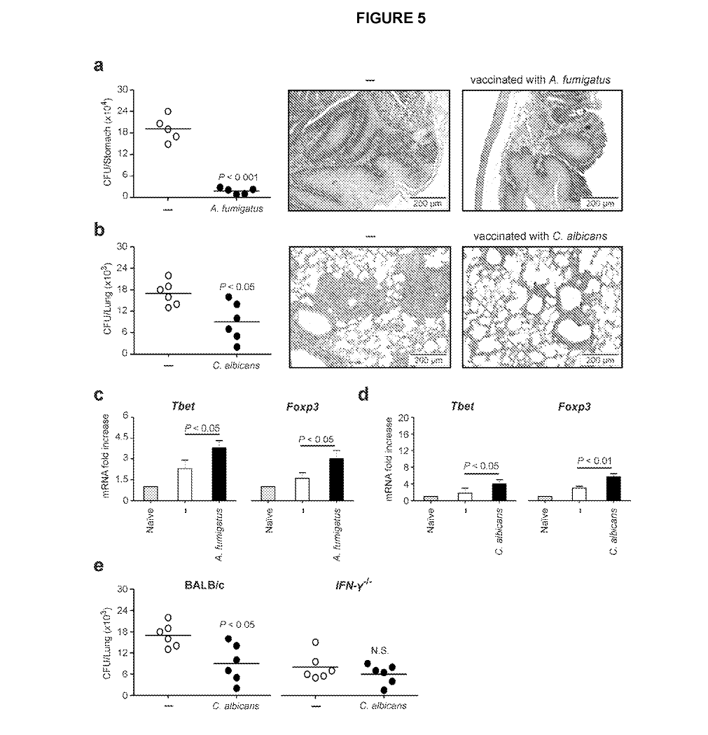

FIG. 5:

Cross-protection between A. fumigatus and C. albicans in mice is mediated by T_{H}1 immunity. (a) Cross-protection by A. fumigatus against gastrointestinal candidiasis. Mice injected with resting A. fumigatus conidia intranasally a week before intragastric injection with C. albicans show reduced inflammatory cell recruitment and acanthosis in comparison to untreated animals. (b) Cross-protection by C. albicans against invasive pulmonary aspergillosis. Mice injected with C. albicans intragastrically a week before intranasal injection of resting A. fumigatus conidia show reduced inflammatory cell recruitment and amelioration of lung inflammatory pathology in comparison to untreated animals. (c) Vaccination with A. fumigatus as well as (d) vaccination with C. albicans showed a significant increase in protective T_{H}1 and regulatory T-cells. (e) Cross-protection between A. fumigatus and C. albicans in mice is dependent on IFN-γ. IFN-γ-deficient mice vaccinated with C. albicans show no reduced fungal growth in the lungs in comparison to untreated animals. Shown are fungal growth (CFU/organ, mean±SE), stomach histology (only a and b) and transcription factor mRNA expression in CD4⁺ T-cells from mesenteric lymph nodes (only a and b) a week after infection with C. albicans. Shown are the results pooled from 2 or 3 experiments in panel a and b, respectively (6 animals/group). Bars indicate SE. P, vaccinated animals versus control.

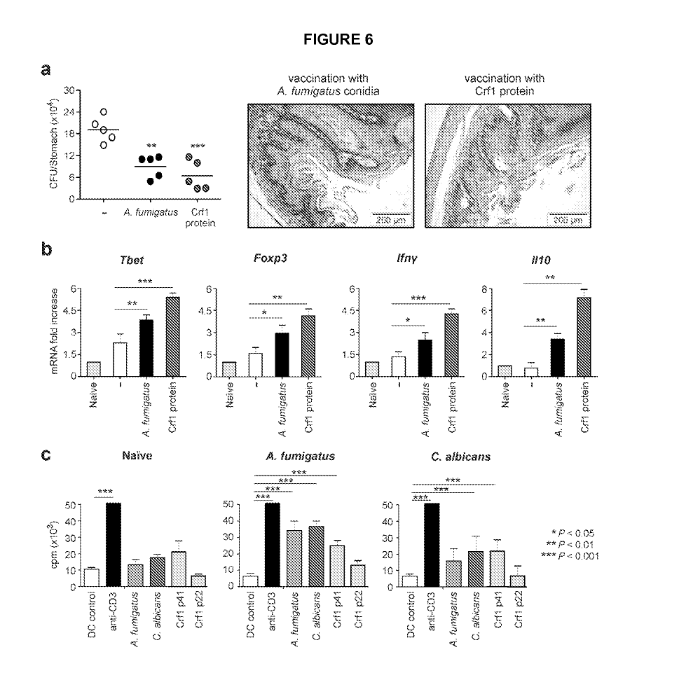

FIG. 6:

A. fumigatus Crf1 protein mediates cross-protection to C. albicans. Mice were injected intranasally with resting A. fumigatus conidia 14 days before infection, or with purified Crf1 protein and murine CpG oligodeoxynucleotide 1862 14, 7 and 3 days before intragastric infection with C. albicans. Note (a) the reduced inflammatory cell recruitment and acanthosis in mice vaccinated with A. fumigatus conidia or Crf1 protein as opposed to control mice shown in FIG. 5a and c and (b) the increase in protective T_{H}1 and regulatory T-cell transcription factors and cytokine levels. Shown are fungal growth (CFU/organ, mean±SE), stomach histology and transcription factor and cytokine mRNA expression in CD4⁺ T-cells from mesenteric lymph nodes a week after infection with C. albicans. Shown are results pooled from 2 experiments (6 animals/group). Bars indicate SE. P, vaccinated animals versus control. (c) Proliferation of purified CD4⁺ spleen cells from untreated mice or animals vaccinated with A. fumigatus or C. albicans in response to control DC, anti-CD3E antibody, heat-inactivated A. fumigatus or C. albicans or A. fumigatus Crf1 p41 and p22 peptide. The results are expressed as mean cpm±SEM of triplicate cultures. P, stimulated versus control DC.

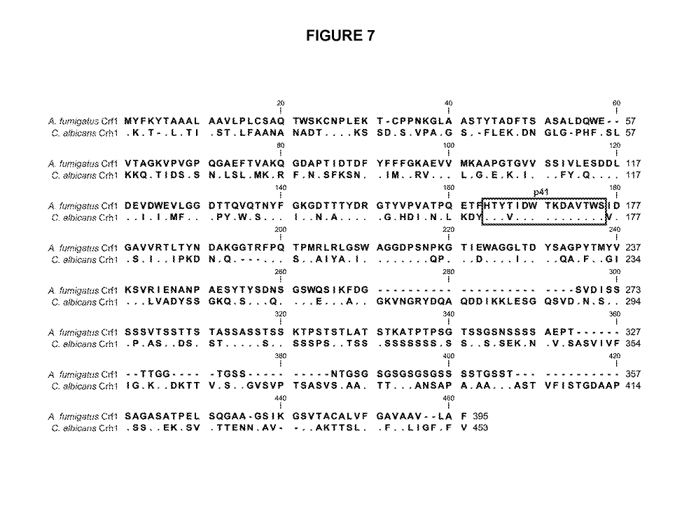

FIG. 7:

Homology between A. fumigatus Crf1 and C. albicans Crh1. Identical residues are denoted as dots, missing residues as hyphen. The A. fumigatus peptide epitope Crf1 p41 is shown in the box.

### EXAMPLE 1

### Cross-Protective T_{H}1 Immunity Against Aspergillus fumigatus and Candida albicans Induced by a Single Peptide

### Results

### The p41 Epitope of the A. Fumigatus Cell Wall Glucanase Crf1 is Highly Immunogenic in Healthy Individuals

As the identification of potential protective fungal antigens is rather intricate due to the extensiveness of fungal genomes, we analyzed 6 different A. fumigatus recombinant proteins based on their previously reported immunogeneic reactivity in mice and humans^{7,10}. Most of the proteins such as superoxide dismutase, catalase, the major allergen and cytotoxin AspF1 and 1,3-beta-glucanosyl-transferase Gel1 elicited only weak interferon-γ (IFN-γ) responses in some donors. By contrast, GPI-anchored extracellular cell wall glucanase Crf1 and secreted protein peptidase induced high IFN-γ in 6 of 7 and 5 of 10 tested donors, respectively. The response was mediated by CD4⁺ T_{H}1 cells, as MHC class II blocking antibody nearly abrogated IFN-γ release (data not shown). Due to the broad response and the sequence similarity to the C. albicans orthologue Crh1, we focused in subsequent experiments on the protein Crf1. By epitope mapping of 7 healthy individuals with different MHC class II alleles, we identified 10 potential candidate epitopes (Table 1). Based on the results obtained by epitope mapping, the probability of antigen presentation by certain MHC class II alleles assessed by epitope prediction¹¹, previously reported immunogeneic reactivity¹² and sequence similarity to the C. albicans homologue Crh1, we then generated CD4⁺ T_{H}1 T-cell clones specific for the peptides p1, p22, p27, p41, p48, p59, p69 and p84 (FIG. 1a and Table 1).

To determine whether the different Crf1 peptide epitopes can be actually processed and presented by dendritic cells (DC), the T-cell clones were stimulated with DC transfected with Crf1 mRNA. All generated p41-specific CD4⁺ T-cell clones were activated readily, whereas only some of the p1-, p22-, p27- and p59-specific CD4⁺ T-cells clones responded to endogenously processed antigen. None of the CD4⁺ T-cell clones specific for the peptides p48, p69 and p84 responded to Crf1 mRNA-transfected DC (FIG. 1b). We next assessed the efficiency of T-cell activation by naturally occurring fungal antigen. As fungal surface structures can influence DC function via engagement of Toll-like and other pattern recognition receptors¹³⁻¹⁵ we first stimulated the T-cell clones with DC incubated with fungal extract to minimize the risk of unspecific T-cell responses. All Crf1 p41-specific T-cell clones of all 5 donors and most Crf1 p1-specific T-cell clones of 2 of 4 donors responded to A. fumigatus extract, whereas only very few T-cell clones specific for Cal p27 and Crf1 p59 and none of the Cal p22-specific T-cell clones could be activated (FIG. 2a). All T-cell clones responding to fungal extract could likewise be activated by inactivated fungus (FIG. 2b). Since A. fumigatus changes its morphology and concurrently also its antigenic properties during conversion from inert fungal spores to invasive pathogenic hyphae¹⁶, we wanted to ascertain which morphotypes of A. fumigatus are recognized by the Crf1 p41-specific T-cell clones. While resting conidia were not able to activate peptide-specific T-cell clones, germinating conidia induced similar T-cell responses as outgrown hyphae (FIG. 2c).

These findings suggest that most healthy donors harbor highly reactive T_{H}1 cells specific for the A. fumigatus Crf1 p41 epitope whereas immunity to all other analyzed peptides seems to be less widespread.

A. fumigatus Crf1 p41-Specific T-Cell Clones are Cross-Reactive to C. albicans

C. albicans is the second most prevalent fungal pathogen in HSCT recipients and the amino acid sequence of the A. fumigatus protein Cal shows some sequence similarity to its homologue Crh1 in C. albicans (FIG. 7). Therefore we verified if the T-cell clones that recognized endogenously processed A. fumigatus are also cross-reactive to C. albicans. All p41-specific T-cell clones of all donors recognized and responded to C. albicans whereas none of the Crf1 p1-, Cal p27- and Cal p59-specific T-cell clones could be activated (FIG. 3a). The A. fumigatus p41 epitope and the corresponding C. albicans epitope are nearly identical differing only in one amino acid, where valine is substituted for threonine at position 4 (FIG. 7). As this observation was made in established strains, we additionally stimulated T-cell clones with different clinical isolates of A. fumigatus and C. albicans and could further confirm this cross-reactivity of the Crf1 p41 epitope (FIG. 3b).

We then analyzed if our T-cell clones might cross-react to other clinically relevant Aspergillus and Candida species such as A. flavus, A. nidulans, A. niger, A. terreus, C. glabrata and C. krusei because they also express proteins with some sequence similarity to A. fumigatus Crf1. With the exception of the Crf1 μl-specific T-cells clones of 1 of 4 donors that showed weak cross-reactivity to A. flavus, A. nidulans and A. terreus, none of the Crf1 p27-, p41- and p59-specific T-cell clones showed any cross-reactivity to these fungi (data not shown). Thus, p41-specific T_{H}1 cells represent a core repertoire in antifungal immune responses being reactive to A. fumigatus but also to C. albicans, the second most important pathogenic fungus in HSCT patients.

Cross-Reactive Crf1 p41-Specific CD4⁺ T_{H}1 Cells are Diverse in their Vβ Repertoire and HLA II Restriction and have a High Proliferative Capacity in Response to Antigen

Since Crf1 p41-specific T-cells can be activated by A. fumigatus as well as C. albicans and all humans are in permanent contact with both pathogens via inhalation of fungal spores or commensalism, we reasoned that the T-cell repertoire of memory cells recognizing this epitope should be broad and polyclonal. We therefore determined the TCR Vβ chains of 7 to 14 Crf1 p41-specific CD4⁺ T clones of each donor by flow cytometry, PCR and nucleotide sequence analysis. The TCRs of each donor belonged to 1 to 5 different Vβ families and showed additional variations on the level of nucleotide sequence in Vβ chains of the same family indicating a diverse repertoire of Crf1 p41-specific T-cells (FIG. 4a).

To evaluate the utility of the Crf1 p41 epitope for potential clinical application, we defined the HLA restriction of Crf1 p41-specific CD4⁺ T clones from different donors by stimulation with peptide-pulsed partially matched LCLs. We found indications that presentation of the Crf1 p41 is mediated by HLA-DRB1*03, 04 and 13 and that in some instances DRB1*03-restricted TCRs might be able to recognize p41 peptide presented by DRB1*13 and vice versa (FIG. 4b). As presentation of the Crf1 p41 peptide could also be mediated by other MHC class II alleles, we verified the HLA restriction by stimulation of PBMC from additional donors. All HLA-DRB1*03, 04 and 13 donors reacted to the Crf1 p41 epitope, while all 5 donors lacking HLA-DRB1*03, 04 and 13 MHC class II alleles did not (data not shown). Further analysis elucidated that the 11-mer peptide HTYTIDWTKDA is the minimal epitope of Crf1 p41 for all 3 HLA alleles (data not shown).

The feasibility of immunotherapeutic strategies such as vaccination or adoptive T-cell transfer is intimately linked to the capacity of antigens to efficiently activate and expand pathogen-specific T-cells. Using an HLA-DRB1*0401-restricted Crf1 p41 tetramer, we identified within the CD4⁺ T-cell fraction 1 to 2 tetramer-positive cells/10⁵ CD4⁺ T-cells (FIG. 4c). Despite this extremely low precursor frequency, we were able to generate Crf1 p41-specific T-cell lines within 14 days with a median percentage of antigen-specific T-cells of 50.6%. These Crf1 p41-specific CD4⁺ T-cell lines produced high amounts of T_{H}1 cytokines and proliferated upon antigen stimulation (FIG. 4c).

Taken together, these results demonstrate that the Crf1 p41 epitope induces an oligoclonal immune response in the great majority of individuals and can be presented by multiple MHC class II alleles. Furthermore, Crf1 p41-specific memory CD4⁺ T-cells possess high proliferative capacity indicating that these cells are not terminally differentiated.

Vaccination with A. fumigatus Conidia or Crf1 Protein Protects Mice Against Subsequent Lethal Infection with C. albicans

To assess the relevance of the cross-reactive T-cell repertoire in vivo, we infected BALB/c mice either intrapulmonary with A. fumigatus or intragastrally with C. albicans a week before re-challenge with C. albicans or A. fumigatus, respectively. The results show that pre-infection with either fungus significantly restricted the fungal growth after rechallenge and ameliorated inflammatory pathology in the respective target organs, the lung and stomach, as judged by the reduced numbers of abscesses, acanthosis and local inflammatory cell recruitment (FIG. 5a,b). Furthermore, pre-infection with A. fumigatus or C. albicans activated Tbet⁺/Foxp3⁺ CD4⁺ T-cells in mesenteric lymph nodes (FIG. 5c,d). Cross-protection by Candida was greatly reduced in IFN-γ-deficient mice further suggesting that protection is mediated by cross-protective T_{H}1 cells (FIG. 5e). To verify whether the Aspergillus versus Candida cross-protection can be mediated by the Aspergillus Crf1 protein, we vaccinated mice with Aspergillus conidia or the Crf1 protein and CpG as adjuvant prior to C. albicans infection. We found that both vaccination strategies reduced the fungal burden and inflammatory pathology in the stomach of mice with candidiasis (FIG. 5a,b and 6a). Again, this correlated with the activation of Tbet⁺/Foxp3⁺ CD4⁺T-cells and their respective cytokines, IFN-γ and IL-10, in mesenteric lymph nodes (FIG. 6b). Antigen-specific proliferation of CD4⁺ spleen cells in untreated mice or animals vaccinated with A. fumigatus or C. albicans suggests that the observed cross-protection could be mediated by T-cells recognizing the same epitope p41 of the Crf1 protein that mediates cross-reactivity in humans. In contrast, the previously described epitope p22¹² did not induce significant proliferation in vaccinated animals (FIG. 6c).

### Discussion

Aspergillus fumigatus and Candida albicans are the two most common fungal pathogens causing severe invasive infections among immunocompromised patients. Although new antifungal drugs for prophylaxis and treatment have reduced incidence and overall mortality of invasive fungal infections, breakthrough infections as well as toxicity counterbalance this development. In this study, we identified the epitope p41 of the A. fumigatus extracellular cell wall glucanase Crf1 as a promising new target for antifungal immunotherapeutic strategies as it induces T_{H}1 cells that are cross-reactive to C. albicans in mice and humans. Moreover, the Crf1 p41 epitope can be presented by at least 3 different MHC class II alleles covering 59.9% of the Caucasian population¹⁷ and activates an oligoclonal T-cell response with a diverse repertoire of TCR Vβ chains. This cross-reactive T-cell epitope is especially interesting for clinical application as Crf1 p41-specific memory CD4⁺ T-cells can be rapidly expanded ex vivo despite low precursor frequencies. Particularly, T-cell mediated cross-reactivity between A. fumigatus and C. albicans seems to be not only restricted to humans but was similarly observed in an animal model, as mice vaccinated with A. fumigatus conidia or Crf1 protein are protected against lethal infection with C. albicans and vice versa, suggesting that T-cell mediated cross-protection could be of significant relevance in antifungal immunity.

Antifungal CD4⁺ T_{H}1 immunity plays a pivotal role in the clearance of most fungal infections including invasive aspergillosis and candidiasis. Immunotherapeutic strategies activating antifungal T_{H}1 CD4⁺ immunity such as adoptive transfer of A. fumigatus-specific CD4⁺ T_{H}1 cells and vaccination with diverse fungal antigens have already shown therapeutic efficacy in mouse models¹⁸ and HSCT recipients². However, potent and safe antigens for broad clinical application are hampered by lack of identification of target epitopes. The A. fumigatus GPI-anchored extracellular cell wall glucanase Crf1 described in our study and by other groups seems to be a potential candidate antigen^{12,18,19}. We could substantiate this observation by identifying T-cells specific for four Crf1 peptide epitopes that can be activated by DC presenting endogenously processed fungal antigen. Out of these four epitopes, the Crf1 p41 epitope was by far the most robust epitope as all derived CD4⁺ T-cell clones from multiple donors responded to this epitope as well as to fungal antigen. On the other hand, we have discovered several other peptides that activated CD4⁺ T-cells which were not-specific for A. fumigatus and did not respond to fungal antigen. This unspecific activation of T-cells is often observed when unphysiologically high doses of peptides are used for T-cell stimulation, but is not indicative of physiologic T-cell activation^{9,20}. In summary, we characterized for the first time CD4⁺ T_{H}1 T-cell clones generated by stimulation with peptides that can be activated by whole A. fumigatus processed by DC.

The Crf1 p41 epitope seems to be an exceptionally potent A. fumigatus antigen, as all T-cell clones showed strong reactivity towards the mould A. fumigatus as well as to the only distantly related yeast C. albicans. The discovery of a defined peptide epitope activating T_{H}1 cells with cross-reactivity to two important fungal pathogens in humans is to our knowledge a novel finding and only little is known in mouse models²¹. The concept of heterologous immunity to different pathogens mediated by cross-reactive T-cells has until now only been described in detail for viral infections²² for instance for influenza virus^{23,24} and recently for flavivirus, the causative agent of dengue fever²⁵⁻²⁷. The discovery of this mechanism has initiated the development of new vaccines, which will target conserved protein domains of different flaviviral strains for induction of cross-protection. In our confirmatory murine experiments, both T_{H}1 associated transcription factors and cytokine mRNA levels as well as experiments with IFN-γ knockout mice suggest that T_{H}1 biased T-cells play indeed a crucial role in mediating cross-protection between the two fungi A. fumigatus and C. albicans^{7,1}. Furthermore, the same epitope mediating cross-reactivity in humans is also cross-reactive in mice. Taken together, our data illustrates for the first time the existence of T-cell based cross-protection between distantly related fungal pathogens in humans as well as mice, which could therefore be considered in general as a strategy for induction and boosting of a broad specific cellular immunity.

Mathematic models have suggested that far more immunogenic T-cell epitopes are existent than TCRs being randomly generated and selected via the thymus in a single individual²⁸. Nevertheless, the adaptive cellular immune response is capable of protecting higher evolutionary organisms from pathogens. This is facilitated on the molecular level between TCR and MHC peptide complexes by several mechanisms which include induced fitting of the TCR, differential TCR docking capabilities, structural degeneracy in the interaction between TCR and MHC complex, molecular mimicry of the epitope and antigen dependent tuning of peptide-MHC flexibility. In line with this theory, the cross-reactivity observed in our study is probably due to molecular mimicry. The A. fumigatus Crf1 epitope p41 and the corresponding epitope of the C. albicans homologue Crh1 differ only in one amino acid (FIG. 7) and both epitopes seem to be recognized by the same TCR. This substitution of an unpolar amino acid for a polar one is most probably located at a non-MHC anchor site since Y163 and D166 represent optimal P1 resp. P4 anchor residues in the epitope binding to HLA-DRB1*03 or DRB1*04. It has been reported that anchor residue substitutions typically have very little negative impact on T-cell reactivity²⁴. In our case, cross-reactivity is observed in spite of a non-anchor residue substitution in P2. This phenomenon was universally observed in different T-cell clones derived from various donors. In contrast, the p41 epitopes of other Aspergillus species differing in 4 to 6 amino acids from the A. fumigatus sequence are no longer cross-reactive. We further observed that CD4⁺ p41-specific HLA-DRB1*03-restricted T cell clones could be activated with peptide presented by HLA-DRB1*13 and vice versa, which is in line with the recently described structural degeneracy of epitopes presented by different MHC class II molecules.

The cross-reactivity of Crf1 p41-specific T-cells to two of the most important fungal pathogens in immunocompromised hosts makes this epitope conceptually an interesting target for immunotherapeutic approaches for prevention and treatment of common fungal infections. The cross-reactivity could be observed in several different isolates implying that the epitope is generally conserved. Notably, as A. fumigatus changes its morphology during germination of inert conidia to outgrown hyphae, the Crf1 p41 epitope is expressed only after germination and during hyphal growth. Therefore, expression of Crf1 occurs in the pathogenic form of the fungus when angio-invasion and destruction of the organ takes place and the cellular immunity is activated. The unresponsiveness to resting conidia could either result from selective expression of Crf1 during fungal growth in accordance with its function in cell wall construction²⁹⁻³¹ or from insufficient T-cell activation due to inadequate DC co-stimulation^{32,33}.

The clinical utility of this epitope is further strengthened because Crf1 p41 T-cells have a broad MHC restriction covering nearly 60% of the Caucasian population and activate an oligoclonal TCR Vβ repertoire in healthy individuals. The feasibility of immunotherapeutic strategies is highly dependent on the capacity of antigens to efficiently expand pathogen-specific memory T-cells. In fact, we were able to demonstrate that the cross-reactive fungal CD4⁺ T-cell populations can be readily expanded in vitro implying that they do not represent terminally differentiated T-cells. This high proliferative capacity is mandatory for immunotherapeutic intervention such as vaccination and adoptive T-cell therapies in immunocompromised hosts for prevention and treatment of common fungal infection.

In summary, we describe a pathogen-specific T-cell response selectively shaped to respond to two common fungal pathogens in humans. This data may have implications for the development of vaccination strategies and T-cell therapy for various infectious diseases.

### Methods

### Cloning, Expression and Purification of Recombinant Proteins

A. fumigatus superoxide dismutase³⁴, catalase³⁵, peptidase³⁶ and 1,3-beta-glucanosyl-transferase Gel1³⁰ were produced in the yeast Pichia pastoris as described³⁷. Extracellular cell wall glucanase Crf1³⁷ (cDNA provided by Utz Reichard) and the major allergen and cytotoxin AspF1¹⁰ were expressed with FLAG-Tags in the human cell line phoenix GALV³⁸.

### Crf1 Peptides

A peptide library covering the complete Crf1 sequence consisting of 94 15-mer peptides overlapping by 4 amino acids (aa) was divided into sub-pools of 10 peptides and 94 single peptides (p1-p94) (PANATecs). 3 11-mer peptides of p40/41 overlapping by 2aa (QETFHTYTIDW, TFHTYTIDWTK, HTYTIDWTKDA) were obtained from JPT Peptide Technologies.

### Isolation of T-Cells and Generation of Dendritic Cells (DC)

Blood was obtained from healthy donors after informed consent. Peripheral blood mononuclear cells (PBMC) were isolated and DC as well as monocytes were generated according to previously published protocols³⁹⁻⁴¹.

### T-Cell Expansion, T-Cell Cloning, Restimulation of T-Cell Clones and Quantification of Cytokines by Interferon-Gamma (IFN-γ) ELISA and ELISPOT Assays

For analysis of recombinant proteins and epitope mapping, T-cell lines were generated by incubation of 1×10⁷ PBMC per well in 6-well cell culture plates with 5 μg/ml protein or peptide for 7d in the presence of 5 U/ml IL-2 (Proleukin, Chiron). Antigen specificity was analyzed by IFN-γ ELISA⁴² after restimulation with autologous monocytes incubated overnight with 5 μg/ml protein in a responder to stimulator (R:S) ratio of 5:1. Epitope mapping was performed by IFN-γ ELISPOT³⁹ after stimulation with peptide-pulsed autologous DC at a R:S ratio of 10:1.

For T-cell expansion, 1×10⁷ PBMC were stimulated with 5 μg/ml peptide in the presence of 5 U/ml IL-2. On d7, T-cells were restimulated with autologous peptide-pulsed monocytes at a R:S ratio of 5:1 and 10 ng/ml IL-7 and IL-15 (R&D systems) were supplemented every other day until d14.

T-cell cloning was performed as described in below. T-cell clones were stimulated either with mature peptide-pulsed (2.5 μg/ml) or Crf1 mRNA-transfected DC, immature DC incubated for 24 h with A. fumigatus cell extract (20 μg/ml) in the presence of cytokine maturation cocktail or immature DC incubated for 24 h with inactivated A. fumigatus or C. albicans fungus (multiplicity of infection 3) in a R:S ratio of 5:1.

### MHC Restriction and T-Cell Receptor (TCR) Repertoire Analysis

HLA restriction was analyzed with partially matched allogeneic EBV-lymphoblastoid cell lines (LCL) (provided by Patricia Comoli and Inge Jedema) pulsed with 10⁻³ to 1 ug/ml peptide using IFN-γ ELISA.

The TCR Vβ repertoire was analyzed flowcytometrically (IOTest Beta Mark Kit, Beckman Coulter), by PCR⁴³ and sequence analysis⁴⁴. A PE-conjugated Crf1 p41-specific HLA-DRB1*0401 MHC class II tetramer was used according to the manufacturers' instructions (Beckman Coulter).

### Animals

Female C57BL/6 Wo kommen die denn vor? and BALB/c mice, 8-10 wk old, were purchased from Charles River (Calco, Italy). Homozygous IFN-γ-deficient mice on BALB/c were bred under specific pathogen-free conditions at the Animal Facility of Perugia University, Perugia. Experiments were performed according to the Italian Approved Animal Welfare Assurance A-3143-01.

C. albicans and A. fumigatus Co-Infection

Mice were infected with 2×10⁷ resting A. fumigatus conidia intranasally^{7,45} and with 10⁸C. albicans intragastrically¹³. For co-infection, mice were infected a week before the subsequent infection. In the vaccination model, mice were injected intranasally with 2×10⁷ Aspergillus resting conidia/20 μl saline once, 14d before infection, or with 5 μg purified Crf1p antigen⁷ and 10 nM murine CpG oligodeoxynucleotide 1862, administered 14 d, 7 d and 3 d before intragastric C. albicans infection⁷. Mice were monitored for survival (MST, days) and fungal growth (CFU/organ expressed as mean±SE). Mice dying of fungal challenge routinely underwent necropsy for histopathological confirmation of infection⁷.

### Real-Time PCR

Real-time RT-PCR was performed from purified CD4⁺ T-cells from thoracic (pulmonary aspergillosis) or mesenteric (gastrointestinal candidiasis) lymph nodes as described^{7,45}.

### Proliferation Assay

DC (1×10⁵) and CD4⁺ T-cells (5×10⁵) were purified from spleens of mice untreated or vaccinated with A. fumigatus or C. albicans, by magnetic separation of MicroBeads (Miltenyi Biotec) and cocultured in round-bottom, 96-well plates in 200 μl complete medium, with and without anti-CD3ε antibody (clone 145-2C11; BD PharMingen), heat-inactivated Aspergillus or Candida (at a cell:fungi ratio of 2:1), 5 μg/mL of Crf1 p41 peptide. Cells were cultured for 3 days at 37° C., 5% CO₂ Eighteen hours before harvesting, cells were pulsed with 0.5 μCi of [³H]thymidine per well. Incorporation into cellular DNA was measured by liquid scintillation counting. The results are expressed as mean cpm±SEM of triplicate cultures.

### Statistical Analysis

Data were analyzed by GraphPad Prism 5.0 program (GraphPad Software). Student's t test or analysis of variance (ANOVA) and Bonferroni's test were used to determine the statistical significance (P) of differences in organ clearance and in vitro assays. The data reported are either from one representative experiment out of three independent experiments or pooled from three experiments. The in vivo groups consisted of 6 mice per group.

### Cloning, Expression and Purification of Recombinant Proteins

We introduced the mutation AspF1 His136Leu to diminish toxicity in the eukaryotic producer cells⁴⁶. Transgene expression of the pcDNA3.1-TOPO vector (Invitrogen) was enhanced with 1 mM sodium butyrate (Sigma-Aldrich)⁴⁷. Producer cells were lysed with 50 mM Tris-HCl pH 7.4, 150 mM NaCl, 1 mM EDTA and 1% Triton X-100 and the recombinant proteins purified under native conditions using M2-agarose according to the manufacturers' instructions (Sigma-Aldrich).

### Fungal Strains and Generation of Fungal Extracts and Inactivated Fungi

Fungal strains A. fumigatus D141, A. nidulans FGSCA4, C. albicans SC5314 and clinical isolates A. fumigatus, A. terreus, A. flavus, A. niger, C. albicans, C. glabrata and C. krusei were provided by Sven Krappmann, Joachim Morschhäuser and Reno Frei.

Fungal extracts were generated as described⁴⁸ and protein content was measured by Bradford protein assay (Perbio Science) according to the manufacturers' instructions. For inactivated fungus Aspergillus conidia were inoculated in growth medium⁴⁹ and cultured for 0, 4, 8 or 12 h. Candida species were cultured for 24 h. Fungi were inactivated with 70% ethanol. Ethanol-inactivated fungi and extracts were validated for sterility by subculturing on Sabouraud dextrose agar.

### T-Cell Cloning

For T-cell cloning 6×10⁷ PBMC were stimulated overnight with 5 μg/ml peptide and 1 μg/ml CD40 antibody (HB14, Miltenyi Biotec). Cells were labelled with anti-CD154⁺ microbeads and isolated by magnetic cell separation according to the manufacturers' instructions (Miltenyi Biotec). Selected cells were cultured with γ-irradiated autologous PBMC (ratio 1:50) and cytokines were supplemented as described above. On d14, T-cell clones were generated by limiting dilution in 96-well plates and expanded using the rapid expansion protocol previously described⁵⁰.

Transfection of Dendritic Cells with Crf1 mRNA

A. fumigatus Crf1 cDNA was cloned into the pGEM4Z vector (provided by Kris Thielemans) and in vitro transcribed using mMESSAGE mMACHINE T Ultra Kit (Ambion) according to the manufacturers' instructions. Crf1 was fused to the N-terminal domain of the invariant chain to enhance antigen presentation by MHC class II^{51,52}. Maturated DC were electroporated with 15 μg mRNA at 300V and 150 μF using the EQUIBIO EasyjecT Plus® apparatus (EQUIBIO) in serum-free medium (OptiMEM, Gibco)⁴¹.

TABLE 1
Immunogeneic peptide epitopes of
A. fumigatus Crf1 tested in 7 donors with
different MHC class II alleles identified by
epitope mapping with IFN-γ ELISPOT.

| No. of | Peptide | aa | |
|---|---|---|---|
| responders | number | positions | Sequence |
| 5 | 41 | 161-175 | HTYTIDWTKDAVTWS |
| | 59 | 233-247 | YTMYVKSVRIENANP |
| | 84 | 333-347 | GSSNTGSGSGSGSGS |
| 4 | 1 | 1-15 | MYFKYTAAALAAVLP |
| | 49 | 193-207 | TRFPQTPMRLRLGSW |
| | 89 | 353-367 | TGSSTSAGASATPEL |
| 3 | 9 | 33-47 | PPNKGLAASTYTADF |
| | 48 | 189-203 | AKGGTRFPQTPMRLR |
| | 58 | 229-243 | SAGPYTMYVKSVRIE |
| | 69 | 273-287 | SSSSVTSSTTSTASS |

### REFERENCES

1. Marr, K. A. Fungal infections in hematopoietic stem cell transplant recipients. Med Mycol 46, 293-302 (2008).
2. Perruccio, K., et al. Transferring functional immune responses to pathogens after haploidentical hematopoietic transplantation. Blood 106, 4397-4406 (2005).
3. Hebart, H., et al. Analysis of T-cell responses to Aspergillus fumigatus antigens in healthy individuals and patients with hematologic malignancies. Blood 100, 4521-4528 (2002).
4. Romani, L. Immunity to fungal infections. Nat Rev Immunol 4, 1-23 (2004).
5. Roilides, E., Sein, T., Schaufele, R., Chanock, S. J. & Walsh, T. J. Increased serum concentrations of interleukin-10 in patients with hepatosplenic candidiasis. J Infect Dis 178, 589-592 (1998).
6. Vultaggio, A., et al. T cells specific for Candida albicans antigens and producing type 2 cytokines in lesional mucosa of untreated HIV-infected patients with pseudomembranous oropharyngeal candidiasis. Microbes Infect 10, 166-174 (2008).
7. Bozza, S., et al. Immune sensing of Aspergillus fumigatus proteins, glycolipids, and polysaccharides and the impact on Th immunity and vaccination. J Immunol 183, 2407-2414 (2009).
8. Capilla, J., Clemons, K. & Stevens, D. The friend of man again? Saccharomyces (S) as a vaccine against invasive aspergillosis. in 47th Intersci. Conf. Antimicrib. Agents Chemother. Abst. G-1708 (Chicago, 2007).
9. Mazza, C. & Malissen, B. What guides MHC-restricted TCR recognition? Semin Immunol 19, 225-235 (2007).
10. Ok, M., et al. Immune responses of human immature dendritic cells can be modulated by the recombinant Aspergillus fumigatus antigen Aspf1. Clin Vaccine Immunol 16, 1485-1492 (2009).
11. Rammensee, H., Bachmann, J., Emmerich, N. P., Bachor, O. A. & Stevanovic, S. SYFPEITHI: database for MHC ligands and peptide motifs. Immunogenetics 50, 213-219 (1999).
12. Zhu, F., Ramadan, G., Davies, B., Margolis, D. A. & Keever-Taylor, C. A. Stimulation by means of dendritic cells followed by Epstein-Barr virus-transformed B cells as antigen-presenting cells is more efficient than dendritic cells alone in inducing Aspergillus f16-specific cytotoxic T cell responses. Clin Exp Immunol 151, 284-296 (2008).
13. Bonifazi, P., et al. Balancing inflammation and tolerance in vivo through dendritic cells by the commensal Candida albicans. Mucosal Immunol 2, 362-374 (2009).
14. Romani, L., et al. Thymosin alpha 1 activates dendritic cells for antifungal Th1 resistance through toll-like receptor signaling. Blood 103, 4232-4239 (2004).
15. Bozza, S., et al. Dendritic cells transport conidia and hyphae of Aspergillus fumigatus from the airways to the draining lymph nodes and initiate disparate Th responses to the fungus. J Immunol 168, 1362-1371 (2002).
16. Latge, J. P. Aspergillus fumigatus and aspergillosis. Clin Microbiol Rev 12, 310-350 (1999).
17. Schipper, R. F., van Els, C. A., D'Amaro, J. & Oudshoorn, M. Minimal phenotype panels. A method for achieving maximum population coverage with a minimum of HLA antigens. Hum Immunol 51, 95-98 (1996).
18. Bozza, S., et al. Vaccination of mice against invasive aspergillosis with recombinant Aspergillus proteins and CpG oligodeoxynucleotides as adjuvants. Microbes Infect 4, 1281-1290 (2002).
19. Chaudhary, N., Staab, J. F. & Marr, K. A. Healthy human T-Cell Responses to Aspergillus fumigatus antigens. PLoS One 5, e9036.
20. Sadegh-Nasseri, S., Dalai, S. K., Korb Ferris, L. C. & Mirshahidi, S. Suboptimal engagement of the T-cell receptor by a variety of peptide-MHC ligands triggers T-cell anergy. Immunology 129, 1-7.
21. Pietrella, D., et al. Mannoprotein from Cryptococcus neoformans promotes T-helper type 1 anticandidal responses in mice. Infect Immun 70, 6621-6627 (2002).
22. Welsh, R. M. & Selin, L. K. No one is naive: the significance of heterologous T-cell immunity. Nat Rev Immunol 2, 417-426 (2002).
23. Lazarski, C. A., Chaves, F. A. & Sant, A. J. The impact of DM on MHC class II-restricted antigen presentation can be altered by manipulation of MHC-peptide kinetic stability. J Exp Med 203, 1319-1328 (2006).
24. Richards, K. A., Chaves, F. A. & Sant, A. J. Infection of HLA-DR1 transgenic mice with a human isolate of influenza a virus (H1N1) primes a diverse CD4 T-cell repertoire that includes CD4 T cells with heterosubtypic cross-reactivity to avian (H5N1) influenza virus. J Virol 83, 6566-6577 (2009).
25. Thomas, S. J., Hombach, J. & Barrett, A. Scientific consultation on cell mediated immunity (CMI) in dengue and dengue vaccine development. Vaccine 27, 355-368 (2009).
26. Trobaugh, D. W., Yang, L., Ennis, F. A. & Green, S. Altered effector functions of virus-specific and virus cross-reactive CD8+ T cells in mice immunized with related flaviviruses. Eur J Immunol 40, 1315-1327.
27. Gao, G., et al., Adenovirus-based vaccines generate cytotoxic T lymphocytes to epitopes of NS1 from dengue virus that are present in all major serotypes. Hum Gene Ther 19, 927-936 (2008).
28. Mason, D. A very high level of crossreactivity is an essential feature of the T-cell receptor. Immunol Today 19, 395-404 (1998).
29. Bruneau, J. M., et al. Proteome analysis of Aspergillus fumigatus identifies glycosylphosphatidylinositol-anchored proteins associated to the cell wall biosynthesis. Electrophoresis 22, 2812-2823 (2001).
30. Mouyna, I., et al. Glycosylphosphatidylinositol-anchored glucanosyltransferases play an active role in the biosynthesis of the fungal cell wall. J Biol Chem 275, 14882-14889 (2000).
31. Schutte, M., et al. Identification of a putative Crf splice variant and generation of recombinant antibodies for the specific detection of Aspergillus fumigatus. PLoS One 4, e6625 (2009).
32. Aimanianda, V., et al. Surface hydrophobin prevents immune recognition of airborne fungal spores. Nature 460, 1117-1121 (2009).
33. Gersuk, G. M., Underhill, D. M., Zhu, L. & Marr, K. A. Dectin-1 and TLRs permit macrophages to distinguish between different Aspergillus fumigatus cellular states. J Immunol 176, 3717-3724 (2006).
34. Holdom, M. D., Lechenne, B., Hay, R. J., Hamilton, A. J. & Monod, M. Production and characterization of recombinant Aspergillus fumigatus Cu,Zn superoxide dismutase and its recognition by immune human sera. J Clin Microbiol 38, 558-562 (2000).
35. Calera, J. A., et al. Cloning and disruption of the antigenic catalase gene of Aspergillus fumigatus. Infect Immun 65, 4718-4724 (1997).
36. Reichard, U., Cole, G. T., Ruchel, R. & Monod, M. Molecular cloning and targeted deletion of PEP2 which encodes a novel aspartic proteinase from Aspergillus fumigatus. Int J Med Microbiol 290, 85-96 (2000).
37. Arroyo, J., et al. The GPI-anchored Gas and Crh families are fungal antigens. Yeast 24, 289-296 (2007).
38. Horn, P. A., Topp, M. S., Morris, J. C., Riddell, S. R. & Kiem, H. P. Highly efficient gene transfer into baboon marrow repopulating cells using GALV-pseudotype oncoretroviral vectors produced by human packaging cells. Blood 100, 3960-3967 (2002).
39. Khanna, N., et al. JC virus-specific immune responses in human immunodeficiency virus type 1 patients with progressive multifocal leukoencephalopathy. J Virol 83, 4404-4411 (2009).
40. Rauser, G., et al. Rapid generation of combined CMV-specific CD4+ and CD8+T-cell lines for adoptive transfer into recipients of allogeneic stem cell transplants. Blood 103, 3565-3572 (2004).
41. Michiels, A., et al. Electroporation of immature and mature dendritic cells: implications for dendritic cell-based vaccines. Gene Ther 12, 772-782 (2005).
42. Diekmann, J., et al. Processing of two latent membrane protein 1 MHC class I epitopes requires tripeptidyl peptidase II involvement. J Immunol 183, 1587-1597 (2009).
43. Schwab, N., et al. CD8+ T-cell clones dominate brain infiltrates in Rasmussen encephalitis and persist in the periphery. Brain 132, 1236-1246 (2009).
44. Monteiro, J., Hingorani, R., Peroglizzi, R., Apatoff, B. & Gregersen, P. K. Oligoclonality of CD8+ T cells in multiple sclerosis. Autoimmunity 23, 127-138 (1996).
45. Bonifazi, P., et al. Intranasally delivered siRNA targeting PI3K/Akt/mTOR inflammatory pathways protects from aspergillosis. Mucosal Immunol 3, 193-205.
46. Kao, R., Shea, J. E., Davies, J. & Holden, D. W. Probing the active site of mitogillin, a fungal ribotoxin. Mol Microbiol 29, 1019-1027 (1998).
47. Palermo, D. P., DeGraaf, M. E., Marotti, K. R., Rehberg, E. & Post, L. E. Production of analytical quantities of recombinant proteins in Chinese hamster ovary cells using sodium butyrate to elevate gene expression. J Biotechnol 19, 35-47 (1991).
48. Braedel, S., et al. Aspergillus fumigatus antigens activate innate immune cells via toll-like receptors 2 and 4. Br J Haematol 125, 392-399 (2004).
49. Kafer, E. Meiotic and mitotic recombination in Aspergillus and its chromosomal aberrations. Adv Genet. 19, 33-131 (1977).
50. Beck, O., at al. Generation of highly purified and functionally active human TH1 cells against Aspergillus fumigatus. Blood 107, 2562-2569 (2006).
51. Malcherek, G., et al. MHC class II-associated invariant chain peptide replacement by T cell epitopes: engineered invariant chain as a vehicle for directed and enhanced MHC class II antigen processing and presentation. Eur J Immunol 28, 1524-1533 (1998).
52. Sanderson, S., Frauwirth, K. & Shastri, N. Expression of endogenous peptide-major histocompatibility complex class II complexes derived from invariant chain-antigen fusion proteins. Proc Natl Acad Sci USA 92, 7217-7221 (1995).
53. Wei, S., Charmley, P., Robinson, M. A. & Concannon, P. The extent of the human germline T-cell receptor V beta gene segment repertoire. Immunogenetics 40, 27-36 (1994).

```
SEQUENCES (SEQ ID Nos)
  1. HTYTIDWTKDAVTWS;
  2. HTYVIDWTKDAVTWS;
  3. HTYVIDWTKDA;
  4. FHTYTIDWTKDAVTW;
  5. PVATPQETFHTYTID;
  6. PQETFHTYTIDWTKD;
  7. TIDWTKDAVTWSIDG;
  8. TKDAVTWSIDGAVVR;
  9. HTYTIDWTKDA;
10. QETFHTYTIDW;
11. TFHTYTIDWTK;
12. YTMYVKSVRIENANP;
13. GSSNTGSGSGSGSGS;
14. MYFKYTAAALAAVLP;
15. TRFPQTPMRLRLGSW;
16. TGSSTSAGASATPEL;
17. PPNKGLAASTYTADF;
18. AKGGTRFPQTPMRLR;
19. SAGPYTMYVKSVRIE;
20. SSSSVTSSTTSTASS;
21. FIG. 7 A. fumigatus Crf1;
22. FIG. 7 C. albicans Crh1.
```

1: A complex comprising a Class II HLA-DRB1*03 or Class II HLA-DRB1*13 molecule bound to a peptide, wherein the peptide comprises the amino acid sequence HTYTIDWTKDAVTWS or a portion thereof, or a variant of the given amino acid sequence or portion wherein the side chains of one or two or three or four or five or six or seven of the amino acid residues are altered, wherein the peptide comprising the portion, or variant, is capable of binding HLA-DRB1*03 and/or HLA-DRB1*13.

2: A complex according to claim 1 which is a soluble complex.

3: A complex according to claim 1 wherein the Class II HLA-DRB1*03 or Class II HLA-DRB1*13 molecule is a synthetic molecule.

4. (canceled)

5: A method for selecting Aspergillus and Candida antigen-specific T cells, the method comprising contacting a population of T cells with a complex according to claim 1.

6: A method according to claim 5 wherein the Class II HLA molecule is present on the surface of an antigen-presenting cell.

7: A method according to claim 5 wherein the Class II HLA molecule is a synthetic soluble Class II HLA molecule.

8: A method according to claim 5 wherein the selected T cell is isolated.

9: A method according to claim 5 wherein the T cell is further selected as being CD3⁺ CD4⁺ or CD3⁺ CD8⁺ or being a T_{H}1 T helper cell or a T regulatory cell.

10: A method according to claim 9 wherein if the T cell is a T_{H}1 helper cell it is subsequently converted to a T regulatory cell.

11: The Aspergillus and Candida antigen-specific T cells obtained by the method of claim 5.

12: Aspergillus and Candida antigen-specific T cells which are able to recognise the HLA-DRB1*03- or HLA-DRB1*13-presented peptide HTYTIDWTKDAVTWS.

13: A T-cell receptor (TCR) or a functionally equivalent molecule to the TCR, which recognises a complex according to claim 1.

14: A polynucleotide encoding a T cell receptor (TCR) as defined in claim 13.

15: An expression vector capable of expressing a T cell receptor (TCR) as defined in claim 13.

16: A host cell, such as a T cell, comprising a polynucleotide according to claim 14.

17: A method of combating Aspergillus or Candida albicans infection in a patient or of combating allergy to Aspergillus or Candida albicans in a patient the method comprising administering to the patient an effective number of Aspergillus and Candida antigen-specific T cells as defined in claim 12.

18. (canceled)

19. (canceled)

20: A method of combating Aspergillus or Candida albicans infection in a patient, the method comprising the steps of (1) obtaining T cells from the patient; (2) introducing into said cells a polynucleotide encoding a T cell receptor (TCR), or a functionally equivalent molecule, as defined in claim 13; (3) introducing the cells produced in step (2) into the patient.

21: A method of combating Aspergillus or Candida albicans infection in a patient which patient carries Class II HLA-DRB1*03 or Class II HLA-DRB1*13, the method comprising the steps of (1) obtaining antigen presenting cells, typically dendritic cells from said patient; (2) exposing said antigen presenting cells ex vivo with a peptide as defined in part (a) of claim 30, wherein the peptide comprising the portion, or variant, is capable of binding HLA-DRB1*03 and/or HLA-DRB1*13; and (3) reintroducing the so treated cells into the patient.

22: A dendritic cell obtained in step (2) of claim 21. Aspergillus infccti n.

23. (canceled)

24. (canceled)

25: An antibody which selectively recognises the peptide HTYTIDWTKDAVTWS.

26. (canceled)

27: A method of combating Aspergillus or Candida albicans infection, or for combating allergy to Aspergillus or Candida albicans, in a patient, the method comprising administering to the patient a therapeutically effective amount of an antibody according to claim 25.

28: A method of determining whether an individual is infected with Aspergillus or Candida albicans, the method comprising determining whether an individual contains Aspergillus and Candida antigen-specific T cells as defined in claim 12.

29: A method of determining whether an individual is infected with Aspergillus or Candida albicans, the method comprising using a TCR according to claim 13.

30: A vaccine against Aspergillus or Candida albicans infection comprising
(a) a peptide comprising the amino acid sequence HTYTIDWTKDAVTWS or a portion thereof, or a variant of the given amino acid sequence or portion wherein the side chains of one or two or three or four or five or six or seven of the amino acid residues are altered, wherein the peptide comprising the portion, or variant, is capable of binding HLA-DRB1*03 and/or HLA-DRB1*04 and/or HLA-DRB1*13, or
(b) a polypeptide which is a fusion of any of the peptides of (a) and another peptide, or
(c) a polynucleotide encoding a peptide of (a) or a polypeptide of (b) or
(d) an expression vector capable of expressing a peptide of (a) or a polypeptide of (b).

31: A vaccine according to claim 30 wherein the peptide has a molecular weight less than 5 000, preferably less than 3 000.

32. (canceled)

33: A method of combating Candida albicans infection in a patient or a method of combating allergy to Candida albicans, the method comprising administering to the patient an effective amount of a peptide or a polypeptide or a polynucleotide or an expression vector as defined in claim 30 wherein the amount of said peptide or polypeptide or amount of said polynucleotide or amount of said expression vector is effective to provoke an anti-Candida albicans response in said patient.

34. (canceled)

35: A vaccine against Aspergillus or Candida albicans infection comprising an antigen presenting cell such as a dendritic cell, which is presenting a peptide or a polypeptide as defined in claim 30.

36. (canceled)

37: A method for combating Candida albicans infection in a patient, the method comprising administering to the patient an effective amount of an antigen presenting cell as defined in claim 35 wherein the amount of said antigen presenting cell which is presenting said peptide or polypeptide is effective to provoke an anti-Candida albicans response in said patient.

38: A method of combating Candida albicans infection in a patient or of combating allergy to Candida albicans in a patient the method comprising administering to the patient an effective number of Aspergillus and Candida antigen-specific T cells which are able to recognise the HLA-presented peptide HTYTIDWTKDAVTWS, or population of T cells which has been activated using the peptide or polypeptide as defined in claim 30.

39: A pharmaceutical composition comprising T cells as defined in claim 38 and a pharmaceutically acceptable carrier.

40-44. (canceled)

45: A method according to claim 17 wherein the Aspergillus and Candida antigen-specific T cell is CD3⁺ CD4⁺.

46. (canceled)

47: A method of combating Candida albicans infection in a patient, the method comprising the steps of (1) obtaining T cells from the patient; (2) introducing into said cells a polynucleotide encoding a T cell receptor (TCR), or a functionally equivalent molecule, obtainable from a T cell as defined in claim 38; (3) introducing the cells produced in step (2) into the patient.

48: A method of combating Candida albicans infection in a patient, the method comprising the steps of (1) obtaining antigen presenting cells, typically dendritic cells from said patient; (2) exposing said antigen presenting cells with a peptide or a polypeptide or a polynucleotide or expression vector as defined in claim 30 ex vivo; and (3) reintroducing the so treated cells into the patient.

49: A pharmaceutical composition comprising a dendritic cell obtained in step (2) of claim 21 and a pharmaceutically acceptable carrier.

50-52. (canceled)

53: A method of combating infection according to claim 17 wherein the patient is receiving allogeneic stem cell transplantation.

54: A method according to claim 46 wherein the regulatory cells are CD25⁺ or Foxp3⁺ or GITR⁺ or CD127⁺.

55. (canceled)

56. (canceled)

57: A peptide of less than 10 000 molecular weight comprising the amino acid sequence HTYTIDWTKDAVTWS or a portion thereof, or a variant of the given amino acid sequence or portion wherein the side chains of one or two or three or four or five or six or seven of the amino acid residues are altered, wherein the peptide comprising the portion, or variant, is capable of binding HLA-DRB1*03 and/or HLA-DRB1*13, provided that the peptide is not a peptide of less than 10 000 molecular weight comprising the amino acid sequence FHTYTIDWTKDAVTW or a portion thereof or a variant of the given amino acid sequence or portion wherein the side chains of one or two or three or four or five or six or seven of the amino acid residues are altered, wherein the peptide comprising the portion, or variant, is capable of binding HLA-DRB1*04.

58: A peptide according to claim 57 which has a molecular weight less than 5 000 or less than 3000.

59. (canceled)

60: A peptide according to claim 57 wherein when bound to HLA-DRB1*03 or HLA-DRB1*13 the peptide-bound HLA-DRB1*03 or HLA-DRB1*13 is capable of identifying Aspergillus and Candida antigen-specific T cells.

61: A peptide according to claim 57 wherein the peptide includes non-peptide bonds.

62: A peptide according to claim 57 wherein the peptide consists of the amino acid sequence HTYTIDWTKDAVTWS or a portion thereof, or a variant of the given amino acid sequence or portion wherein the side chains of one or two or three or four or five or six or seven of the amino acid residues are altered, wherein the portion or variant is capable of binding HLA-DRB1*03 and/or HLA-DRB1*13, provided that the peptide is not a peptide of less than 10 000 molecular weight comprising the amino acid sequence FHTYTIDWTKDAVTW or a portion thereof or a variant of the given amino acid sequence or portion wherein the side chains of one or two or three or four or five or six or seven of the amino acid residues are altered, wherein the peptide comprising the portion, or variant, is capable of binding HLA-DRB1*04.

63: A peptide according to claim 62 wherein the peptide consists of the amino acid sequence HTYTIDWTKDAVTWS.

64: A polypeptide which is a fusion of the peptide of claim 57 and another peptide.

65: A polypeptide according to claim 64 which comprises an HLA heavy chain molecule joined via a flexible linker to a peptide according to any one of the preceding claims wherein the said peptide is able to occupy the peptide-binding groove of the HLA molecule.

66: A polypeptide according to claim 64 wherein the other peptide is an invariant chain of an HLA molecule and wherein the peptide comprising the amino acid sequence HTYTIDWTKDAVTWS or a portion or a variant thereof is able to occupy the peptide-binding groove of the HLA molecule.

67: A polynucleotide encoding a peptide or a polypeptide comprising the peptide of claim 57.

68. (canceled)

69: An expression vector comprising the polynucleotide of claim 67.

70. (canceled)

71: A method of producing a peptide or a polypeptide, the method comprising culturing the host cell according to claim 70 and obtaining the peptide or polypeptide from the host cell or its culture medium.

72: A pharmaceutical composition comprising a peptide or a polypeptide comprising the peptide of claim 57 and a pharmaceutically acceptable carrier.

73: A pharmaceutical composition comprising a polynucleotide according to claim 67 and a pharmaceutically acceptable carrier.

74. (canceled)

75. (canceled)

76: A method of combating Aspergillus or Candida albicans infection in a patient or a method of combating allergy to Aspergillus or Candida albicans, the method comprising administering to the patient an effective amount of a peptide or a polypeptide comprising the peptide of claim 57 or a polynucleotide encoding said peptide or polypeptide, wherein the amount of said peptide or polypeptide or amount of said polynucleotide is effective to provoke an anti-Aspergillus or anti-Candida albicans response in said patient.

77. (canceled)

78. (canceled)

79: An antigen presenting cell, such as a dendritic cell, which is presenting a peptide or a polypeptide comprising the peptide of claim 57.

80: A vaccine against Aspergillus or Candida albicans infection comprising an antigen presenting cell according to claim 79.

81. (canceled)

82: A method for combating Aspergillus or Candida albicans infection in a patient, the method comprising administering to the patient an effective amount of an antigen presenting cell according to claim 79 wherein the amount of said antigen presenting cell which is presenting said peptide or polypeptide is effective to provoke an anti-Aspergillus or anti-Candida albicans response in said patient.

83: A method for selecting Aspergillus and Candida antigen-specific T cells, the method comprising contacting a population of T cells with a peptide or a polypeptide comprising the peptide of claim 57 or a polynucleotide encoding said peptide or polypeptide.

84: Aspergillus and Candida antigen-specific T cells obtainable by the method of claim 83 wherein the Aspergillus and Candida antigen-specific T cells recognises human Class II MHC molecules expressed on the surface of an antigen-presenting cell and loaded with said peptide or polypeptide.

85: A T-cell receptor (TCR) which detects Aspergillus or Candida albicans infection, the TCR being obtainable from the Aspergillus and Candida antigen-specific T cells of claim 84, or a functionally equivalent molecule to the TCR, wherein the TCR, or a functionally equivalent molecule to the TCR, recognises human Class II MHC molecules expressed on the surface of an antigen-presenting cell and loaded with said peptide or polypeptide.

86: A method of combating Aspergillus or Candida albicans infection in a patient or of combating allergy to Aspergillus or Candida albicans in a patient the method comprising administering to the patient an effective population of T cells which has been activated using a peptide or a polypeptide comprising the peptide of claim 57.

87. (canceled)

88. (canceled)

89: A method of combating Aspergillus or Candida albicans infection in a patient, the method comprising the steps of (1) obtaining T cells from the patient; (2) introducing into said cells a polynucleotide encoding a T cell receptor (TCR), or a functionally equivalent molecule, as defined in claim 85; (3) introducing the cells produced in step (2) into the patient.

90: A method of combating Aspergillus or Candida albicans infection in a patient, the method comprising the steps of (1) obtaining antigen presenting cells, typically dendritic cells from said patient; (2) exposing said antigen presenting cells ex vivo with a peptide or a polypeptide comprising the peptide of claim 57 or with a polynucleotide encoding said peptide or polypeptide; and (3) reintroducing the so treated cells into the patient.

91-95. (canceled)
